(19) United States
(12) Patent Application Publication
(10) Pub. No.: US 2016/0257676 A1
(43) Pub. Date: Sep. 8, 2016

(54) Aminoisoquinoline Derivatives as Protein Kinase Inhibitors

(71) Applicant: ETERNITY BIOSCIENCE INC., Cranbury, NJ (US)

(72) Inventors: Minsheng Zhang, Warren, NJ (US); Dong Liu, Bridgewater, NJ (US); Biao Lu, Shanghai (CN)

(73) Assignee: ETERNITY BIOSCIENCE INC., Cranbury, NJ (US)

(21) Appl. No.: 14/428,183

(22) PCT Filed: Sep. 12, 2013
(86) PCT No.: PCT/US13/59362

§ 371 Date: Mar. 13, 2015

Related U.S. Application Data
(60) Provisional application No. 61/701,155, filed on Sep. 14, 2012.

(51) Int. Cl. C07D 471/04; C12N 9/12; A61K 31/4745; A61K 45/06; C07D 217/22; A61K 31/472

(52) U.S. Cl. CPC C07D 471/04; C07D 217/22; A61K 31/472; A61K 31/4745; A61K 45/06; C12N 9/12; C12Y 207/11001

(57) ABSTRACT

The present invention provides novel aminoisoquinoline compounds as defined in the specification, compositions thereof use of these compounds as protein kinase inhibitors and as therapeutic agents for treatment of Raf kinase, in particular BRAF^{V600E} kinase, related diseases or disorders, such as cancers. In addition, the invention also includes methods and processes for preparing these novel aminoisoquinoline compounds.

### CROSS REFERENCE TO RELATED APPLICATIONS

This application claims priority under 35 U.S.C. §119(e) to U.S. Provisional Application No. 61/701,155, filed on Sep. 14, 2012, which is incorporated herein by reference in its entirety.

### FIELD OF THE INVENTION

The present invention relates to novel aminoisoquinoline derivatives, and compositions thereof, useful for the treatment of hyperproliferative diseases, such as various cancers, melanomas and leukemia.

### BACKGROUND OF THE INVENTION

Kinases are a superfamily of enzymes that transfer a phosphate group from ATP to target proteins. There are more than 518 kinases encoded in the human genome, including 90 tyrosine kinases, 388 serine/threnine kinases and 40 atypical kinases (Manning, G., et al., Science, 2002, 298(5600): 1912-1934). They play vital roles in cell activation, proliferation, differentiation, migration, vascular permeability, and so on. Dysfunction of kinases has been implicated in various diseases such as cancer, inflammation, cardiovascular diseases, diabetes, and neuronal disorders. Several kinase inhibitors have been developed for the treatment of cancers, including but not limited to imatinib, dasatinib, nilotinib, gefitinib, erlotinib, lapatinib, sunitinib, sorafenib, pazopanib, evrolimus, trastuzumab, cetuximab, panitumumab, and bevacizumab (Knight, Z. A., et al., Nat. Rev. Cancer, 2010, 10(2): 130-137).

BRAF is a member of the Raf kinase family of serine/threonine-specific protein kinases. BRAF plays an important role in regulating the MAPK/ERK signaling pathway, which affects cell division, proliferation, differentiation, and secretion. The RAS/RAF/MEK/ERK pathway acts as a signal transducer to send extracellular signals such as hormones, cytokines, and various growth factors into cell nucleus, directing a range of biochemical and physiological processes including cell differentiation, proliferation, growth, and apoptosis (McCubrey, J. A., et al., Biochim. Biophys. Acta, 2007, 1773 (8): 1263-84). The RAS/RAF/MEK/ERK pathway is frequently mutated in many human cancers (Downward, J., Nat. Rev. Cancer, 2003, 3 (1): 11-22). The finding that mutations in BRAF caused a wide range of human cancers and many of these tumors are dependent on the constitutive activation of BRAF/MEK/ERK pathway fueled drug discovery efforts in searching for small molecule inhibitors targeting BRAF mutants (especially the most common form of BRAF^{V600E}) (Davies, H., et al., Nature, 2002, 417: 949-954) (Flaherty, K. T., et al., New Engl. J. Med., 2010, 363: 809-819). It was found that BRAF mutations are responsible for more than 50% of malignant melanomas, ˜45% of papillary thyroid cancer, 10% of colorectal cancers, and had also been identified in ovarian, breast, and lung cancers (Cantwell-Dorris, E. R., et al., Molecular Cancer Therapy, 2011, 10: 385-394). Recently it was reported that almost all hairy-cell leukemia patients carry BRAF^{V600E} mutation and inhibition of the enzyme caused significant remission of the disease (Sascha, D., et al., New Engl. J. Med., 2012, 366:2038-2040). BRAF-specific inhibitors such as Vemurafenib (RG7204), PLX-4720, GDC-0879, and Dabrofenib (GSK2118436) have been reported to be efficacious in causing tumor regression in both preclinical and clinical studies (Flaherty, K. T., et al., New Engl. J. Med., 2010, 363: 809-819; Kefford, R. A., et al., J. Clin. Oncol., 2010, 28: 15s).

Accordingly, the identification and development of small-molecules that specifically modulate BRAF^{V600E} kinase activity will serve as therapeutic approaches for successful treatment of a variety of BRAF^{V600E} kinase-related diseases or disorders, such as cancers.

### SUMMARY OF THE INVENTION

This invention provides novel aminoquinoline derivatives as useful Raf kinase, in particular BRAF^{v600E}, inhibitors and as new therapeutic agents for BRAF^{V600E} kinase-related hyperproliferative diseases or disorders, such as cancers, including but not limited to, melanomas, papillary thyroid cancer, colorectal ovarian, breast, and lung cancers, and certain types of leukemia.

In one aspect, the present invention provides a compound of formula (I):

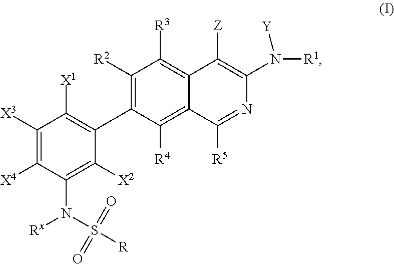

or a tautomer, a prodrug, or a pharmaceutically acceptable salt or solvate thereof, wherein:

Y is hydrogen or C₁-C₄ alkyl, and Z is selected from hydrogen, halogen, C₁-C₄ alkyl, C₁-C₄ haloalkyl, C₁-C₄ alkoxy, C₁-C₄ haloalkoxy, and —NR^{a}R^{b}; or alternatively, Y and Z are connected through a double bond (“Z═Y”) and are each independently CR^{y}, CR^{z}, or nitrogen (N), wherein R^{y} and R^{z} are each independently selected from hydrogen, halogen, hydroxyl, C₁-C₄ alkyl, C₃-C₆ cycloalkyl, C₁-C₄ haloalkyl, C₁-C₄ alkoxy, and C₁-C₄ haloalkoxy;

X¹, X², X³, and X⁴ are each independently selected from hydrogen, halogen, hydroxyl, C₁-C₄ alkyl, C₁-C₄ haloalkyl, C₁-C₄ alkoxy, and C₁-C₄ haloalkoxy;

R is selected from C₁-C₆ alkyl, C₃-C₆ cycloalkyl, C₆-C₁₀ aryl, and 5- to 10-membered heteroaryl, 5- to 10-membered heterocyclyl, C₃-C₆ cycloalkyl-(C₁-C₄)-alkyl, C₆-C₁₀ aryl-(C₁-C₄)-alkyl, 5- to 10-membered heteroaryl-(C₁-C₄)-alkyl, and 5- to 10-membered heterocyclyl-(C₁-C₄)-alkyl, each optionally substituted with one, two, or three substituents independently selected from halogen, hydroxyl, C₁-C₄ alkyl, C₁-C₄ haloalkyl, C₁-C₄ alkoxy, C₁-C₄ haloalkoxy, —NR^{c}R^{d}, cyano, nitro, oxo, —C(O)R⁶, —C(O)OR⁷, and —C(O)NR^{c}R^{d};

R^{x} is hydrogen or C₁-C₄ alkyl, or alternatively, R^{x} and R, together with the nitrogen (N) and sulfur (S) atoms to which they are attached, form a five- or six-membered ring;

R¹ is hydrogen, C₁-C₆ alkyl, C₆-C₁₀ aryl, benzyl, —C(O)R⁶, or —C(O)OR⁷, each optionally substituted with one, two or three substituents independent selected from halogen, C₁-C₄ alkyl, haloalkyl, C₁-C₄ alkoxy, C₁-C₄ alkoxy, cyano, and NR^{a}R^{b};

R², R³, R⁴, and R⁵ are each independently hydrogen, halogen, C₁-C₄ alkyl, C₁-C₄ haloalkyl, C₁-C₄ alkoxy, and C₁-C₄ haloalkoxy;

R^{a} and R^{b} are each independently selected from hydrogen, C₁-C₆ alkyl, benzyl, and —C(O)OR⁷, and

R⁶ is hydrogen or C₁-C₄ alkyl;

R⁷ is C₁-C₄ alkyl; and

R^{c} and R^{d} are each independently hydrogen or C₁-C₄ alkyl.

In one embodiment of this aspect, the invention provides compounds according to formula (I), wherein Y is hydrogen or C₁-C₄ alkyl, and Z is selected from hydrogen, halogen, C₁-C₄ alkyl, C₁-C₄ haloalkyl, C₁-C₄ alkoxy, C₁-C₄ haloalkoxy, and —NR^{a}R^{b}, wherein R^{a}, R^{b}, R¹-R⁵, R, R^{x}, and X¹-X⁴ are defined as above.

In another embodiment of this aspect, the invention provides compounds according to formula (I), wherein Z and Y are connected through a double bond (Z═Y) and are each independently CR^{y}, CR^{z}, or nitrogen (N), further characterized by formula (II):

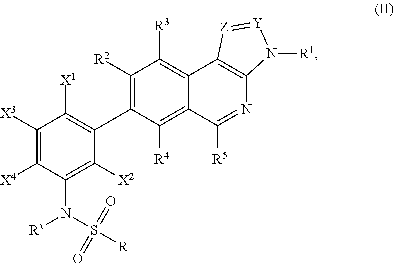

or a tautomer, a prodrug, or a pharmaceutically acceptable salt or solvate thereof, wherein R¹-R⁵, R, R^{x}, R^{y}, R^{z}, and X¹-X⁴ are defined as above.

In another aspect, the present invention provides a composition comprising a compound according to formula (I) or (II) as defined above, or a tautomer, a prodrug, or a pharmaceutically acceptable salt or solvate thereof. In one embodiment of this aspect, the composition further contains a pharmaceutically acceptable carrier.

In another aspect, the present invention provides a method of treating a hyperproliferative disease or disorder, comprising administering to a patient in need thereof a therapeutically effective amount of a compound according to formula (I) or (II) as defined above, or a tautomer, a prodrug, or a pharmaceutically acceptable salt or solvate thereof. The compound can be administered in a composition further comprising a pharmaceutically acceptable carrier.

In another aspect, the present invention provides use of a compound according to formula (I) or (II) as defined above for manufacture of a medicament for treatment of a hyperproliferative disease or disorder. The hyperproliferative disease or disorder is preferably associated with Raf kinase, in particular BRAF^{V600E} kinase, activities, such as a cancer. The hyperproliferative disease or disorder is preferably selected from melanomas; papillary thyroid, colorectal, ovarian, breast, and lung cancers; and leukemia.

In another aspect, the present invention provides an in vitro method of modulating BRAF^{V600E} kinase activity, the method comprising contacting a tissue culture comprising BRAF^{V600E} kinase with a compound according to formula (I) or (II) as defined above.

Other embodiments of the present invention also include methods of synthesizing a compound according to formula (I) or (II) as defined above, including but not limited to the exemplified compounds, as essentially described and shown.

Other aspects and embodiments of the present invention will be better appreciated through the following description and examples.

### DETAILED DESCRIPTION OF THE INVENTION

The present invention provides novel aminoisoquinoline compounds, compositions thereof, use of these compounds as BRAF^{V600E} inhibitors and as therapeutic agents for treatment of Raf kinase, in particular BRAF^{V600E} kinase, related diseases or disorders, as well as methods of synthesizing these novel compounds.

In one aspect, the present invention provides a compound of formula (I):

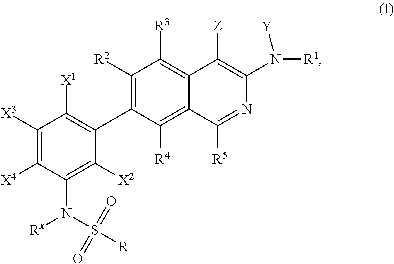

or a tautomer, a prodrug, or a pharmaceutically acceptable salt or solvate thereof, wherein:

Y is hydrogen or C₁-C₄ alkyl, and Z is selected from hydrogen, halogen, C₁-C₄ alkyl, C₁-C₄ haloalkyl, C₁-C₄ alkoxy, C₁-C₄ haloalkoxy, and —NR^{a}R^{b}; or alternatively, Y and Z are connected through a double bond (“Z═Y”) and are each independently CR^{y}, CR^{z}, or nitrogen (N), wherein R^{y} and R^{z} are each independently selected from hydrogen, halogen, hydroxyl, C₁-C₄ alkyl, C₃-C₆ cycloalkyl, C₁-C₄ haloalkyl, C₁-C₄ alkoxy, and C₁-C₄ haloalkoxy;

X¹, X², X³, and X⁴ are each independently selected from hydrogen, halogen, hydroxyl, C₁-C₄ alkyl, C₁-C₄ haloalkyl, C₁-C₄ alkoxy, and C₁-C₄ haloalkoxy;

R is selected from C₁-C₆ alkyl, C₃-C₆ cycloalkyl, C₆-C₁₀ aryl, and 5- to 10-membered heteroaryl, 5- to 10-membered heterocyclyl, C₃-C₆ cycloalkyl-(C₁-C₄)-alkyl, C₆-C₁₀ aryl-(C₁-C₄)-alkyl, 5- to 10-membered heteroaryl-(C₁-C₄)-alkyl, and 5- to 10-membered heterocyclyl-(C₁-C₄)-alkyl, each optionally substituted with one, two, or three substituents independently selected from halogen, hydroxyl, C₁-C₄ alkyl, C₁-C₄ haloalkyl, C₁-C₄ alkoxy, C₁-C₄ haloalkoxy, —NR^{c}R^{d}, cyano, nitro, oxo, —C(O)R⁶, —C(O)OR⁷, and —C(O)NR^{c}R^{d};

R^{x} is hydrogen or C₁-C₄ alkyl, or alternatively, R^{x} and R, together with the nitrogen (N) and sulfur (S) atoms to which they are attached, form a five- or six-membered ring;

R¹ is hydrogen, C₁-C₆ alkyl, C₆-C₁₀ aryl, benzyl, —C(O)R⁶, or —C(O)OR⁷, each optionally substituted with one, two or three substituents independent selected from halogen, C₁-C₄ alkyl, haloalkyl, C₁-C₄ alkoxy, C₁-C₄ alkoxy, cyano, and NR^{a}R^{b};

R², R³, R⁴, and R⁵ are each independently hydrogen, halogen, C₁-C₄ alkyl, C₁-C₄ haloalkyl, C₁-C₄ alkoxy, and C₁-C₄ haloalkoxy;

R^{a} and R^{b} are each independently selected from hydrogen, C₁-C₆ alkyl, benzyl, and —C(O)OR⁷, and

R⁶ is hydrogen or C₁-C₄ alkyl;

R⁷ is C₁-C₄ alkyl; and

R^{c} and R^{d} are each independently hydrogen or C₁-C₄ alkyl.

In one embodiment of this aspect, the invention provides compounds according to formula (I), wherein Y is hydrogen or C₁-C₄ alkyl, and Z is selected from hydrogen, halogen, C₁-C₄ alkyl, C₁-C₄ haloalkyl, C₁-C₄ alkoxy, C₁-C₄ haloalkoxy, and —NR^{a}R^{b}.

In another embodiment of this aspect, the invention provides compounds according to formula (I), wherein Y is hydrogen, and Z is hydrogen.

In another embodiment of this aspect, the invention provides compounds according to formula (I), wherein R¹ is hydrogen or C₁-C₆ alkyl optionally substituted with —NR^{a}R^{b}, wherein R^{a} and R^{b} are independently selected from hydrogen and —C(O)OR⁷.

In another embodiment of this aspect, the invention provides compounds according to formula (I), wherein R is selected from C₁-C₆ alkyl, C₃-C₆ cycloalkyl, and C₆-C₁₀ aryl, each optionally substituted with one, two, or three substituents independently selected from halogen, C₁-C₄ alkyl, C₁-C₄ haloalkyl, C₁-C₄ alkoxy, and C₁-C₄ haloalkoxy.

In another embodiment of this aspect, the invention provides compounds according to formula (I), wherein X¹, X², X³, and X⁴ are independently hydrogen or halogen; R², R³, R⁴, and R⁵ are each independently hydrogen, C₁-C₄ alkyl, C₁-C₄ alkoxy, or C₁-C₄ haloalkoxy; and R is C₁-C₆ alkyl optionally substituted with one, two, or three substituents independently selected from halogen and C₁-C₄ alkoxy.

In another embodiment of this aspect, the invention provides compounds according to formula (I), wherein:
Y and Z are each hydrogen;
X¹ and X² are each independently fluoro (F) or chloro (Cl);
X³ and X⁴ are each hydrogen;
R¹ is hydrogen or C₁-C₆ alkyl optionally substituted by —NHCOOR⁷, wherein R⁷ is C₁-C₄ alkyl;
R² is hydrogen, C₁-C₄ alkoxy, or C₁-C₄ haloalkoxy;
R³, R⁴, and R⁵ are each hydrogen;
R^{x} is hydrogen; and
R is C₁-C₆ alkyl optionally substituted by one to three halogen atoms.

In another embodiment of this aspect, the invention provides a compound selected from the group consisting of:
N-[3-(3-amino-7-isoquinolyl)-2,4-difluoro-phenyl]propane-1-sulfonamide;
methyl N-[(1S)-2-[[7-[2,6-difluoro-3-(propylsulfonylamino)phenyl]-3-isoquinolyl]amino]-1-methyl-ethyl]carbamate;
N-[3-(3-amino-6-methoxy-7-isoquinolyl)-2,4-difluoro-phenyl]propane-1-sulfonamide;
methyl N-[(1S)-2-[[7-[2,6-difluoro-3-(propylsulfonylamino)phenyl]-6-methoxy-3-isoquinolyl]amino]-1-methyl-ethyl]carbamate;
methyl N-[(1R)-2-[[7-[2,6-difluoro-3-(propylsulfonylamino)phenyl]-6-methoxy-3-isoquinolyl]amino]-1-methyl-ethyl]carbamate;
methyl N-[(1S)-2-[[7-[2,6-difluoro-3-(propylsulfonylamino)phenyl]-6-(2-fluoroethoxy)-3-isoquinolyl]amino]-1-methyl-ethyl]carbamate; and
methyl N-[(1S)-2-[[7-[2,6-difluoro-3-(propylsulfonylamino)phenyl]-6-ethyl-3-isoquinolyl]amino]-1-methyl-ethyl]carbamate.

In another embodiment of this aspect, the invention provides compounds according to formula (I), wherein Z and Y are connected through a double bond (Z═Y) and are each independently CR^{y}, CR^{z}, or nitrogen (N), further characterized by formula (II):

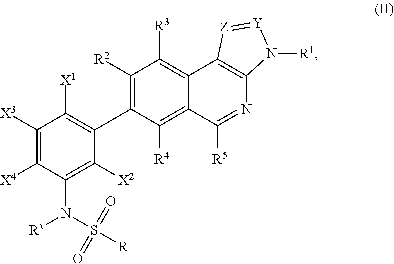

or a tautomer, a prodrug, or a pharmaceutically acceptable salt or solvate thereof.

In another embodiment of this aspect, the invention provides compounds according to formula (II), wherein R¹ is hydrogen, —C(O)R⁶, or C₁-C₆ alkyl optionally substituted with —NR^{a}R^{b}, wherein R^{a} and R^{b} are independently selected from hydrogen and —C(O)OR⁷.

In another embodiment of this aspect, the invention provides compounds according to formula (II), wherein R is selected from C₁-C₆ alkyl, C₃-C₆ cycloalkyl, and C₆-C₁₀ aryl, each optionally substituted with one, two, or three substituents independently selected from halogen, C₁-C₄ alkyl, C₁-C₄ haloalkyl, C₁-C₄ alkoxy, and C₁-C₄ haloalkoxy.

In another embodiment of this aspect, the invention provides compounds according to formula (II), wherein X¹, X², X³, and X⁴ are independently hydrogen or halogen; R², R³, R⁴, and R⁵ are each independently hydrogen, C₁-C₄ alkyl, C₁-C₄ alkoxy, C₁-C₄ haloalkoxy; and R is C₁-C₆ alkyl optionally substituted with one, two, or three substituents independently selected from halogen and C₁-C₄ alkoxy.

In another embodiment of this aspect, the invention provides compounds according to formula (II), wherein R^{x} and R, together with the nitrogen (N) and sulfur (S) atoms to which they are attached, form a five- or six-membered ring.

In another embodiment of this aspect, the invention provides compounds according to formula (II), wherein R^{x} and R together form —CH₂CH₂CH₂—.

In another embodiment of this aspect, the invention provides compounds according to formula (II), wherein:
X¹ and X² are each independently fluoro (F) or chloro (Cl);
X³ and X⁴ are each hydrogen;
R¹ is hydrogen or C₁-C₆ alkyl optionally substituted by —NHCOOR⁷, wherein R⁷ is C₁-C₄ alkyl;
R² is hydrogen, C₁-C₄ alkoxy, or C₁-C₄ haloalkoxy;
R³, R⁴, and R⁵ are each hydrogen;
R^{x} is hydrogen;
R is C₁-C₆ alkyl optionally substituted by one to three halogen atoms;
R^{y} and R^{z} are each independently selected from hydrogen, halogen, C₁-C₄ alkyl, and C₃-C₆ cycloalkyl.

In another embodiment of this aspect, the invention provides compounds according to formula (II), wherein Y is nitrogen (N) and Z is C—R^{z}, further characterized by formula (IIa):

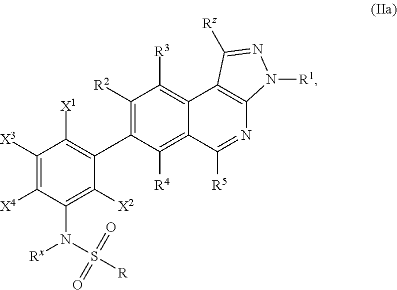

or a tautomer, a prodrug, or a pharmaceutically acceptable salt or solvate thereof, wherein R^{z} is selected from hydrogen, halogen, C₁-C₄ alkyl, C₃-C₆ cycloalkyl, C₁-C₄ haloalkyl, C₁-C₄ alkoxy, and NR^{a}R^{b}.

In another embodiment of this aspect, the invention provides compounds according to formula (IIa), wherein R¹ is hydrogen, —C(O)R⁶, or C₁-C₆ alkyl optionally substituted with —NR^{a}R^{b}, wherein R^{a} and R^{b} are independently selected from hydrogen and —C(O)OR⁷.

In another embodiment of this aspect, the invention provides compounds according to formula (IIa), wherein R is selected from C₁-C₆ alkyl, C₃-C₆ cycloalkyl, and C₆-C₁₀ aryl, each optionally substituted with one, two, or three substituents independently selected from halogen, C₁-C₄ alkyl, C₁-C₄ haloalkyl, C₁-C₄ alkoxy, and C₁-C₄ haloalkoxy.

In another embodiment of this aspect, the invention provides compounds according to formula (IIa), wherein X¹, X², X³, and X⁴ are independently hydrogen or halogen; R², R³, R⁴, and R⁵ are each independently hydrogen, C₁-C₄ alkyl, C₁-C₄ alkoxy, or C₁-C₄ haloalkoxy; and R is C₁-C₆ alkyl optionally substituted with one, two, or three substituents independently selected from halogen and C₁-C₄ alkoxy.

In another embodiment of this aspect, the invention provides compounds according to formula (IIa), wherein R^{x} and R, together with the nitrogen (N) and sulfur (S) atoms to which they are attached, form a five- or six-membered ring.

In another embodiment of this aspect, the invention provides compounds according to formula (IIa), wherein R^{x} and R together form —CH₂CH₂CH₂—.

In another embodiment of this aspect, the invention provides compounds according to formula (IIa), wherein:
X¹ and X² are each independently fluoro (F) or chloro (Cl);
X³ and X⁴ are each hydrogen;
R¹ is hydrogen or C₁-C₆ alkyl optionally substituted by —NHCOOR⁷, wherein R⁷ is C₁-C₄ alkyl;
R² is hydrogen, C₁-C₄ alkoxy, or C₁-C₄ haloalkoxy;
R³, R⁴, and R⁵ are each hydrogen;
R^{x} is hydrogen;
R is C₁-C₆ alkyl optionally substituted by one to three halogen atoms;
R^{z} is selected from hydrogen, halogen, C₁-C₄ alkyl, and C₃-C₆ cycloalkyl.

In another embodiment of this aspect, the invention provides compounds according to formula (II), wherein Y is C—R^{y} and Z is nitrogen (N), further characterized by formula (IIb):

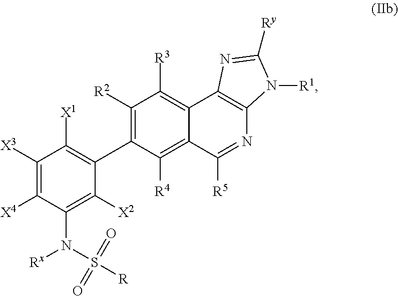

or a tautomer, a prodrug, or a pharmaceutically acceptable salt or solvate thereof, wherein R^{y} is selected from hydrogen, halogen, C₁-C₄ alkyl, C₃-C₆ cycloalkyl, C₁-C₄ haloalkyl, C₁-C₄ alkoxy, and NR^{a}R^{b}.

In another embodiment of this aspect, the invention provides compounds according to formula (IIb), wherein R¹ is hydrogen, —C(O)R⁶, or C₁-C₆ alkyl optionally substituted with —NR^{a}R^{b}, wherein R^{a} and R^{b} are independently selected from hydrogen and —C(O)OR⁷.

In another embodiment of this aspect, the invention provides compounds according to formula (IIb), wherein R is selected from C₁-C₆ alkyl, C₃-C₆ cycloalkyl, and C₆-C₁₀ aryl, each optionally substituted with one, two, or three substituents independently selected from halogen, C₁-C₄ alkyl, C₁-C₄ haloalkyl, C₁-C₄ alkoxy, and C₁-C₄ haloalkoxy.

In another embodiment of this aspect, the invention provides compounds according to formula (IIb), wherein X¹, X², X³, and X⁴ are independently hydrogen or halogen; R², R³, R⁴, and R⁵ are each independently hydrogen, C₁-C₄ alkyl, C₁-C₄ alkoxy, C₁-C₄ haloalkoxy; and R is C₁-C₆ alkyl optionally substituted with one, two, or three substituents independently selected from halogen and C₁-C₄ alkoxy.

In another embodiment of this aspect, the invention provides compounds according to formula (IIb), wherein:
X¹ and X² are each independently fluoro (F) or chloro (Cl);
X³ and X⁴ are each hydrogen;
R¹ is hydrogen or C₁-C₆ alkyl optionally substituted by —NHCOOR⁷, wherein R⁷ is C₁-C₄ alkyl;
R² is hydrogen, C₁-C₄ alkoxy, or C₁-C₄ haloalkoxy;
R³, R⁴, and R⁵ are each hydrogen;
R^{x} is hydrogen;
R is C₁-C₆ alkyl optionally substituted by one to three halogen atoms;
R^{y} is selected from hydrogen, halogen, C₁-C₄ alkyl, and C₃-C₆ cycloalkyl.

In another embodiment of this aspect, the invention provides compounds according to formula (II), wherein Y is C—R^{y} and Z is C—R^{z}, further characterized by formula (IIc):

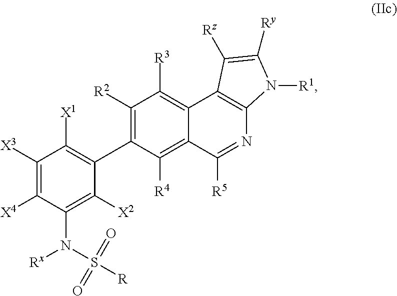

or a tautomer, a prodrug, or a pharmaceutically acceptable salt or solvate thereof, wherein R^{y} and R^{z} are each independently selected from hydrogen, halogen, C₁-C₄ alkyl, C₃-C₆ cycloalkyl, C₁-C₄ haloalkyl, C₁-C₄ alkoxy, and NR^{a}R^{b}.

In another embodiment of this aspect, the invention provides compounds according to formula (IIc), wherein R¹ is hydrogen, —C(O)R⁶, or C₁-C₆ alkyl optionally substituted with —NR^{a}R^{b}, wherein R^{a} and R^{b} are independently selected from hydrogen and —C(O)OR⁷.

In another embodiment of this aspect, the invention provides compounds according to formula (IIc), wherein R is selected from C₁-C₆ alkyl, C₃-C₆ cycloalkyl, and C₆-C₁₀ aryl, each optionally substituted with one, two, or three substituents independently selected from halogen, C₁-C₄ alkyl, C₁-C₄ haloalkyl, C₁-C₄ alkoxy, and C₁-C₄ haloalkoxy.

In another embodiment of this aspect, the invention provides compounds according to formula (IIc), wherein X¹, X², X³, and X⁴ are independently hydrogen or halogen; R², R³, R⁴, and R⁵ are each independently hydrogen, C₁-C₄ alkyl, C₁-C₄ alkoxy, C₁-C₄ haloalkoxy; and R is C₁-C₆ alkyl optionally substituted with one, two, or three substituents independently selected from halogen and C₁-C₄ alkoxy.

In another embodiment of this aspect, the invention provides compounds according to formula (IIc), wherein:
X¹ and X² are each independently fluoro (F) or chloro (Cl);
X³ and X⁴ are each hydrogen;
R¹ is hydrogen or C₁-C₆ alkyl optionally substituted by —NHCOOR⁷, wherein R⁷ is C₁-C₄ alkyl;
R² is hydrogen, C₁-C₄ alkoxy, or C₁-C₄ haloalkoxy;
R³, R⁴, and R⁵ are each hydrogen;
R^{x} is hydrogen;
R is C₁-C₆ alkyl optionally substituted by one to three halogen atoms;
R^{y} and R^{z} are each independently selected from hydrogen, halogen, C₁-C₄ alkyl, and C₃-C₆ cycloalkyl.

In another embodiment of this aspect, the invention provides a compound selected from the group consisting of:
N-[2,4-difluoro-3-(3H-pyrazolo[3,4-c]isoquinolin-7-yl)phenyl]propane-1-sulfonamide;
N-[3-(1-bromo-3H-pyrazolo[3,4-c]isoquinolin-7-yl)-2,4-difluoro-phenyl]propane-1-sulfonamide;
N-[3-(1-cyclopropyl-3H-pyrazolo[3,4-c]isoquinolin-7-yl)-2,4-difluoro-phenyl]propane-1-sulfonamide;
N-[2-chloro-4-fluoro-3-(3H-pyrazolo[3,4-c]isoquinolin-7-yl)phenyl]propane-1-sulfonamide;
N-[3-(1-bromo-3H-pyrazolo[3,4-c]isoquinolin-7-yl)-2-chloro-4-fluoro-phenyl]propane-1-sulfonamide;
N-[2-chloro-3-(1-cyclopropyl-3H-pyrazolo[3,4-c]isoquinolin-7-yl)-4-fluoro-phenyl]propane-1-sulfonamide;
N-[2,4-difluoro-3-(3H-pyrazolo[3,4-c]isoquinolin-7-yl)phenyl]-3-fluoro-propane-1-sulfonamide;
N-[2-chloro-4-fluoro-3-(3H-pyrazolo[3,4-c]isoquinolin-7-yl)phenyl]-3-fluoro-propane-1-sulfonamide;
N-[2,4-dichloro-3-(3H-pyrazolo[3,4-c]isoquinolin-7-yl)phenyl]propane-1-sulfonamide;
N-[4-chloro-2-fluoro-3-(3H-pyrazolo[3,4-c]isoquinolin-7-yl)phenyl]propane-1-sulfonamide;
2-[2,4-difluoro-3-(3H-pyrazolo[3,4-c]isoquinolin-7-yl)phenyl]-1,2-thiazolidine 1,1-dioxide;
N-[2,4-difluoro-3-(8-methoxy-3H-pyrazolo[3,4-c]isoquinolin-7-yl)phenyl]propane-1-sulfonamide;
N-[2-chloro-4-fluoro-3-(8-methoxy-3H-pyrazolo[3,4-c]isoquinolin-7-yl)phenyl]propane-1-sulfonamide;
N-[2,4-difluoro-3-(8-methoxy-3H-pyrazolo[3,4-c]isoquinolin-7-yl)phenyl]-3-fluoro-propane-1-sulfonamide;
N-[2-chloro-4-fluoro-3-(8-methoxy-3H-pyrazolo[3,4-c]isoquinolin-7-yl)phenyl]-3-fluoro-propane-1-sulfonamide;
N-[3-(1-cyclopropyl-8-methoxy-3H-pyrazolo[3,4-c]isoquinolin-7-yl)-2,4-difluoro-phenyl]propane-1-sulfonamide;
N-[2-chloro-3-(1-cyclopropyl-8-methoxy-3H-pyrazolo[3,4-c]isoquinolin-7-yl)-4-fluoro-phenyl]propane-1-sulfonamide;
N-[2-chloro-3-(1-cyclopropyl-8-methoxy-3H-pyrazolo[3,4-c]isoquinolin-7-yl)-4-fluoro-phenyl]-3-fluoro-propane-1-sulfonamide;
N-[3-(1-cyclopropyl-8-methoxy-3H-pyrazolo[3,4-c]isoquinolin-7-yl)-2,4-difluoro-phenyl]-3-fluoro-propane-1-sulfonamide;
N-[2,4-difluoro-3-(3H-pyrrolo[2,3-c]isoquinolin-7-yl)phenyl]propane-1-sulfonamide; and
N-[2,4-difluoro-3-(3H-imidazo[4,5-c]isoquinolin-7-yl)phenyl]propane-1-sulfonamide,

or a tautomer, a prodrug, a pharmaceutically acceptable salt or solvate thereof.

In another aspect, the present invention provides a composition comprising a compound according to any of formulae (I), (II), (IIa), (IIb) and (IIc) as defined according to any of the embodiments described above, or a tautomer, a prodrug, or a pharmaceutically acceptable salt or solvate thereof. In one embodiment of this aspect, the composition further contains a pharmaceutically acceptable carrier.

In another aspect, the present invention provides a method of treating a hyperproliferative disease or disorder, comprising administering to a mammalian patient in need thereof a therapeutically effective amount of a compound according to any of formulae (I), (II), (IIa), (IIb) and (IIc) as defined according to any of the embodiments described above, or a tautomer, a prodrug, or a pharmaceutically acceptable salt or solvate thereof.

In another aspect, the present invention provides a method of treating a hyperproliferative disease or disorder, comprising administering to a patient in need thereof a composition comprising a compound according to any of formulae (I), (II), (IIa), (IIb) and (IIc) as defined according to any of the embodiments described above, or a tautomer, a prodrug, or a pharmaceutically acceptable salt or solvate thereof. In one embodiment of this aspect, the composition further contains a pharmaceutically acceptable carrier.

In another aspect, the present invention provides use of a compound according to any of formulae (I), (II), (IIa), (IIb) and (IIc) as defined in any of the embodiments described above for manufacture of a medicament for treatment of a hyperproliferative disease or disorder.

In another aspect, the present invention provides a compound according to any of formulae (I), (II), (IIa), (IIb) and (IIc) as defined in any of the embodiments described above for treatment of a hyperproliferative disease or disorder selected from melanomas; papillary thyroid, colorectal, ovarian, breast, and lung cancers; and leukemia.

In one embodiment, the hyperproliferative disease or disorder treated according to the present invention is a cancer.

In another embodiment, the hyperproliferative disease or disorder is selected from melanomas; papillary thyroid, colorectal, ovarian, breast, and lung cancers; and leukemia.

In another embodiment, the method of treating a hyperproliferative disease or disorder further includes administering to the patient a therapeutically effective amount of a second therapeutic agent.

In another embodiment, the second therapeutic agent is a different anticancer agent.

In one embodiment, the patient is a mammalian animal, including but not limited to humans, dogs, horses, etc. Preferably the patient is a human.

In another aspect, the present invention provides an in vitro method of modulating BRAF^{V600E} kinase activity, the method comprising contacting a tissue culture comprising BRAF^{V600E} kinase with a compound according to any of formulae (I), (II), (IIa), (IIb) and (IIc) as defined in any of the embodiments described above

Other embodiments of the present invention also include methods of synthesizing a compound according to any of formulae (I), (II), (IIa), (IIb) and (IIc) as defined in any of the embodiments described above, including but not limited to the exemplified compounds, as essentially described and shown.

Yet other aspects and embodiments may be found in the description provided herein.

Any terms in the present application, unless specifically defined, will take the ordinary meanings as understood by a person of ordinary skill in the art.

As used herein, the singular forms “a”, “an”, and “the” include plural reference unless the context clearly dictates otherwise.

Unless stated otherwise, all aryl, cycloalkyl, heteroaryl, and heterocyclyl groups of the present disclosure may be substituted as described in each of their respective definitions. For example, the aryl part of an arylalkyl group such as benzyl may be substituted as described in the definition of the term “aryl.”

The term “alkoxy,” as used herein, refers to an alkyl group attached to the parent molecular moiety through an oxygen atom. Representative examples of alkoxy group include, but are not limited to, methoxy (CH₃O—), ethoxy (CH₃CH₂O—), and t-butoxy ((CH₃)₃CO—).

The term “alkyl,” as used herein, refers to a group derived from a straight or branched chain saturated hydrocarbon by removal of a hydrogen from one of the saturated carbons. The alkyl group preferably contains from one to ten carbon atoms. Representative examples of alkyl group include, but are not limited to, methyl, ethyl, isopropyl, and tert-butyl.

The term “aryl,” as used herein, refers to a group derived from an aromatic carbocycle by removal of a hydrogen atom from an aromatic ring. The aryl group can be monocyclic, bicyclic or polycyclic. Representative examples of aryl groups include phenyl and naphthyl.

The term “benzyl,” as used herein, refers to a methyl group on which one of the hydrogen atoms is replaced by a phenyl group, wherein said phenyl group may be substituted by one or more substituents. Representative examples of benzyl group include, but are not limited to, PhCH₂—, 4-MeO—C₆H₄CH₂—, and 2,4,6-tri-methyl-C₆H₄CH₂—.

The term “cyano,” as used herein, refers to —CN.

The term “cycloalkyl,” as used herein, refers to a group derived from a monocyclic saturated carbocycle, having preferably three to eight carbon atoms, by removal of a hydrogen atom from the saturated carbocycle. Representative examples of cycloalkyl groups include, but are not limited to, cyclopropyl, cyclopentyl, cyclohexyl.

The terms “halo” and “halogen,” as used herein, refer to F, Cl, Br, or I.

The term “haloalkoxy,” as used herein, refers to a haloalkyl group attached to the parent molecular moiety through an oxygen atom.

The term “haloalkyl,” as used herein, refers to an alkyl group substituted by at least one halogen atom. The haloalkyl group can be an alkyl group of which all hydrogen atoms are substituted by halogens. Representative examples of haloalkyl include, but are not limited to, trifluoromethyl (CF₃—), 1-chloroethyl (ClCH₂CH₂—), and 2,2,2-trifluoroethyl (CF₃CH₂—).

The term “heteroaryl,” as used herein, refers to a group derived from a monocyclic or bicyclic compound comprising at least one aromatic ring comprising one or more, preferably one to three, heteroatoms independently selected from nitrogen, oxygen, and sulfur, by removal of a hydrogen atom from the aromatic ring. As is well known to those skilled in the art, heteroaryl rings have less aromatic character than their all-carbon counterparts. Thus, for the purposes of the invention, a heteroaryl group need only have some degree of aromatic character. Illustrative examples of heteroaryl groups include, but are not limited to, pyridyl, pyridazinyl, pyrimidyl, pyrazyl, triazinyl, pyrrolyl, pyrazolyl, imidazolyl, pyrimidinyl, furyl, thienyl, isoxazolyl, thiazolyl, isoxazolyl, oxazolyl, indolyl, quinolinyl, isoquinolinyl, benzisoxazolyl, benzothiazolyl, and benzothienyl.

The term “heterocyclyl,” as used herein, refers to a group derived from a monocyclic or bicyclic compound comprising at least one nonaromatic ring comprising one or more, preferably one to three, heteroatoms independently selected from nitrogen, oxygen, and sulfur, by removal of a hydrogen atom from the nonaromatic ring. The heterocyclyl groups of the present disclosure can be attached to the parent molecular moiety through a carbon atom or a nitrogen atom in the group. Examples of heterocyclyl groups include, but are not limited to, morpholinyl, oxazolidinyl, piperazinyl, piperidinyl, pyrrolidinyl, tetrahydrofuryl, thiomorpholinyl, and indolinyl.

The terms “hydroxy” or “hydroxyl,” as used herein, refer to —OH.

The term “nitro,” as used herein, refers to —NO₂.

The term “oxo,” as used herein, refers to “═O”.

The compounds of the present disclosure can exist as pharmaceutically acceptable salts or solvates. The term “pharmaceutically acceptable salt,” as used herein, means any non-toxic salt that, upon administration to a recipient, is capable of providing the compounds or the prodrugs of a compound of this invention. The salts can be prepared during the final isolation and purification of the compounds or separately by reacting a suitable nitrogen atom with a suitable acid. Acids commonly employed to form pharmaceutically acceptable salts include inorganic acids such as hydrochloric acid, hydrobromic acid, hydroiodic acid, sulfuric acid, phosphoric acid, hydrogen bisulfide as well as organic acids, such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, acetic acid acid, and related inorganic and organic acids.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include, but are not limited to, lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, and N-methylmorpholine.

The term “solvate,” as used herein, means a physical association of a compound of this invention with one or more, preferably one to three, solvent molecules, whether organic or inorganic. This physical association includes hydrogen bonding. In certain instances the solvate will be capable of isolation, for example when one or more, preferably one to three, solvent molecules are incorporated in the crystal lattice of the crystalline solid. Exemplary solvates include, but are not limited to, hydrates, ethanolates, methanolates, and isopropanolates. Methods of solvation are generally known in the art.

The term “therapeutically effective amount,” as used herein, refers to the total amount of each active component that is sufficient to show a meaningful patient benefit, e.g., a sustained reduction in viral load. When applied to an individual active ingredient, administered alone, the term refers to that ingredient alone. When applied to a combination, the term refers to combined amounts of the active ingredients that result in the therapeutic effect, whether administered in combination, serially, or simultaneously.

The term “pharmaceutically acceptable,” as used herein, refers to those compounds, materials, compositions, and/or dosage forms which are, within the scope of sound medical judgment, suitable for use in contact with the tissues of patients without excessive toxicity, irritation, allergic response, or other problem or complication commensurate with a reasonable benefit/risk ratio, and are effective for their intended use.

The term “patient” includes both human and other mammals.

The term “treating” refers to: (i) preventing a disease, disorder or condition from occurring in a patient that may be predisposed to the disease, disorder, and/or condition but has not yet been diagnosed as having it; (ii) inhibiting the disease, disorder, or condition, i.e., arresting its development; and (iii) relieving the disease, disorder, or condition, i.e., causing regression of the disease, disorder, and/or condition.

### Synthetic Methods

The compounds of the present invention can be prepared in a number of ways known to one skilled in the art of organic synthesis. The compounds of the present invention can be synthesized using the methods described below, together with synthetic methods known in the art of synthetic organic chemistry, or by variations thereon as appreciated by those skilled in the art. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the invention.

Abbreviations or terms used in the following synthetic schemes or processes take the meanings as commonly understood by those skilled in the art.

### Synthetic Schemes

Aryl bromide piece bearing sulfonamide was synthesized from corresponding aniline with sulfonyl chloride. The aniline was made according to known literature from commercially available material.

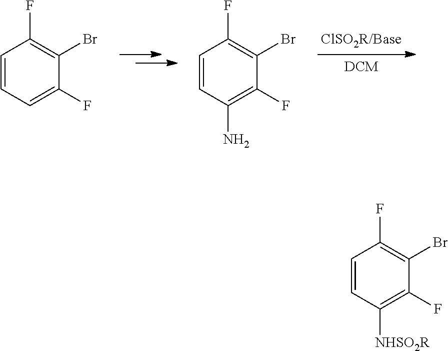

The chemistry used to synthesize analogues A is described in Scheme 1. Bromo-substituted benzyl nitrile was reduced to its benzylic amine, which was condensed with acetimidate to afford acetimidamide intermediate. The subsequent ring closure reaction was carried out in conc. sulfuric acid to give the isoquinoline fragment, which was converted to corresponding boronic ester via Miyaura reaction. The boronic ester was coupled with aryl bromide to give biaryl intermediate, followed by reductive amination to get analogues A.

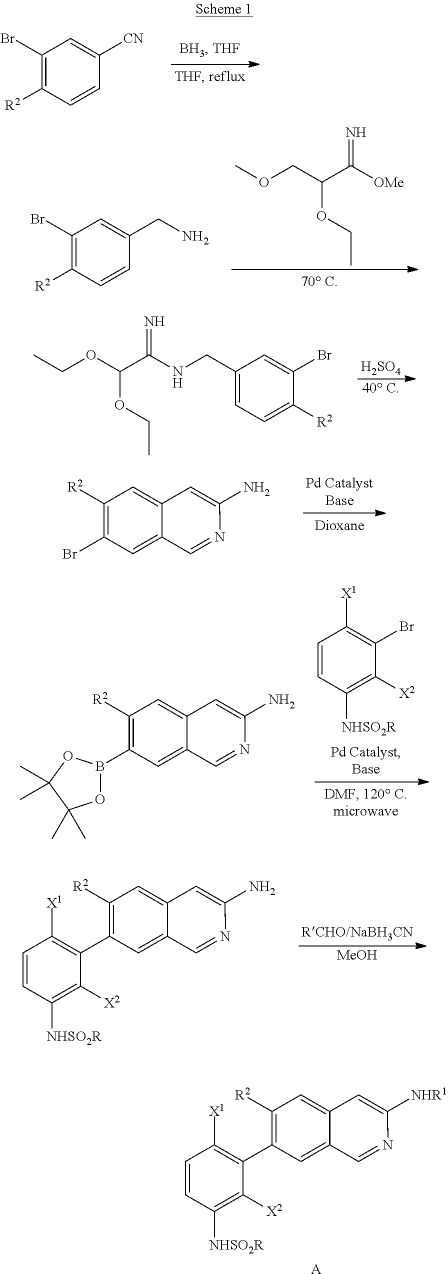

The chemistry used to synthesize analogues B is described in Scheme 2. 4-Bromo-benzylic nitrile was condensed with paraformaldehde in the presence of acids to give lactam intermediate, which was converted to isoquinoline piece via Vilsmeier-Haack reaction followed by oxidation with potassium permanganate. The ortho-chloro aryl aldehyde was condensed with 1.5 eq of hydrazine to give its hydrazone intermediate followed by heating in hydrazine as solvent to afford 7-bromo-3H-pyrazolo[3,4-c]isoquinoline. The bromo derivative was converted to its boronic ester which was protected with acyl group on pyrazole and further coupled with another bromo fragment to give biaryl intermediate via Suzuki reaction in microwave conditions (common thermal condition doesn't work). Selective bromination on pyrazole ring and second Suzuki reaction gave analogues B.

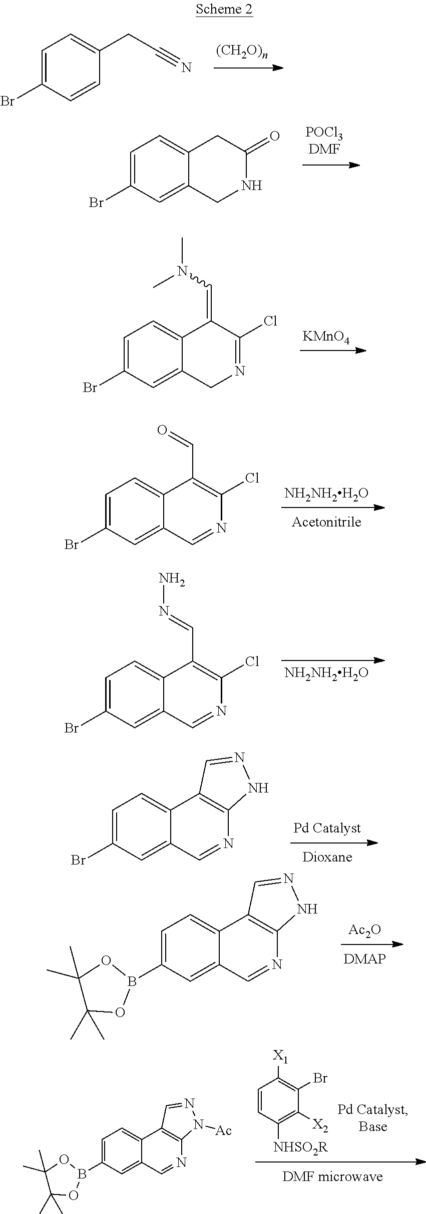

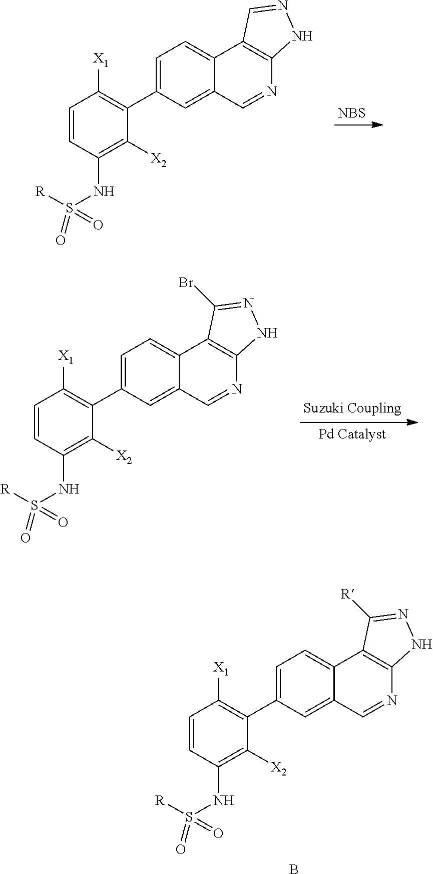

The chemistry used to synthesize analogues C is described in Scheme 3. Bromine substituted benzoic acid was reduced to its benzylic alcohol, followed by bromination and cyanation afforded benzylic nitrile intermediate. After treated with base it was condensed with methyl formic ester or acyl chloride smoothly to give functionalized nitrile which was converted to benzylic pyrazole intermediate. The subsequent assembly of the isoquinoline ring was done through Pictet-spengler type reaction. The resulting bromo intermediate was converted to its boronic ester which was further coupled with another bromo fragment to give biaryl intermediate via Suzuki reaction. Finally, debenzylation was done by palladium-mediated hydrogenolysis to yield analogues C.

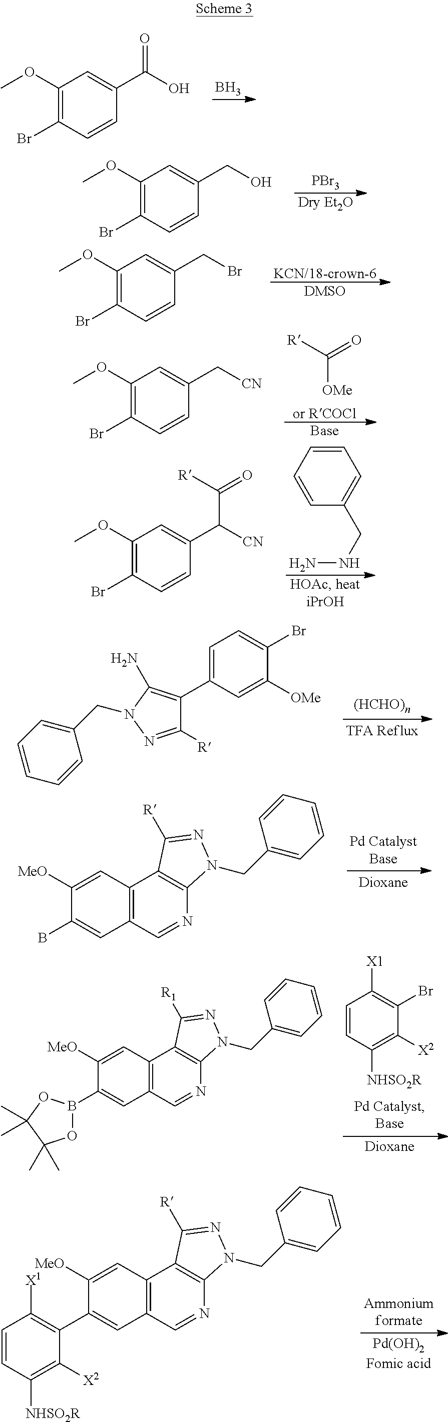

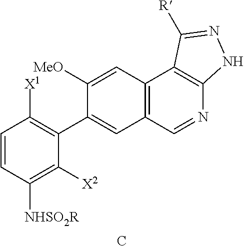

The chemistry used to synthesize analogues D is described in Scheme 4. It started from bromo substituted isoquinoline which was converted to its iodide intermediate with NIS. After coupling with TMS acetylene via Sonogashira reaction and simple acyl protection, it was converted easily to azaindole intermediate with TBAF as base. Consecutive palladium catalyzed reactions provided analogues D.

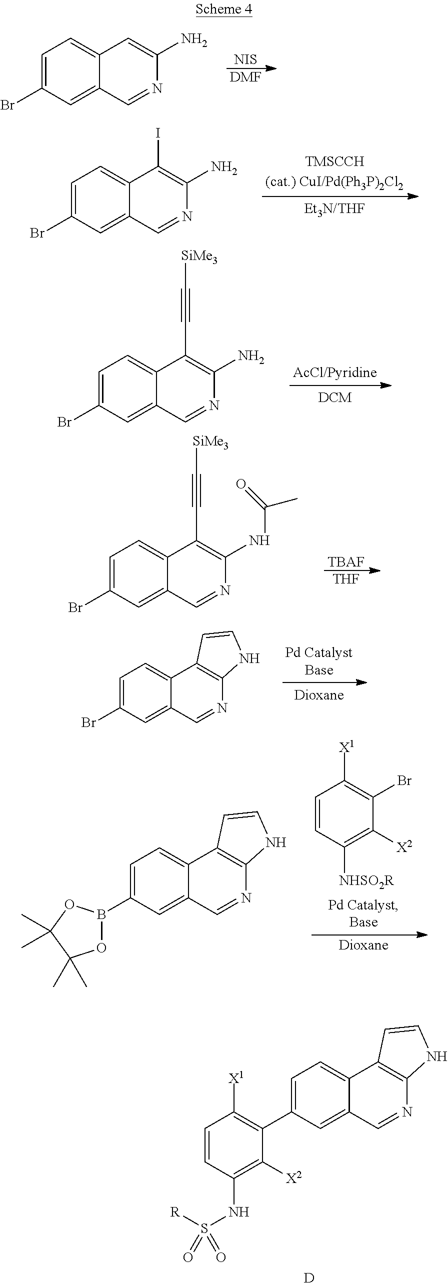

The chemistry used to synthesize analogues E is described in Scheme 5. It started from bromo substituted isoquinoline which was converted to diamine intermediate by nitration and reduction. After condensed with triethyl orthoformate in formic acid, it was converted easily to its imidazole intermediate which went through similar procedures as in scheme 2 to afford analogues E.

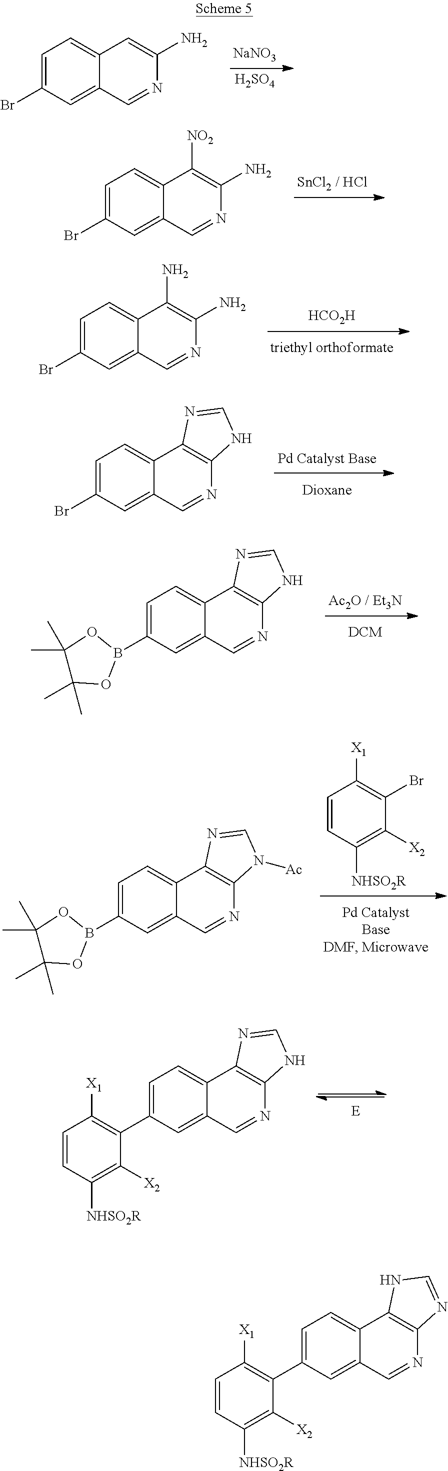

### Biological Assays

BRAF^{V600E} Enzymatic Activity Assay:

The BRAF^{V600E} enzymatic assay was performed using a LanthaScreen kinase assay kit purchased from Life Technologies (Grand Island, N.Y.). The assay was conducted according to the procedure provided in the assay kit. In brief, the enzyme reaction was carried out in the kinase reaction buffer containing BRAF^{V600E} (20 ng/mL), ATP (2 μM), Fluorescein-MAP2K1 inactivesubstrate (0.4 μM), HEPES (50 mM, pH 7.5), 0.01% BRIJ-35, MgCl₂ (10 mM), and EGTA (1 mM) in the presence or absence of the tested articles at various concentrations in 384-well plate at room temperature (22±1° C.) for 60 minutes. The final reaction volume for each reaction was 10 μl. The reaction was stopped by addition of 10 μl of TR-FRET dilution buffer supplemented with kinase quench buffer (10 mM EDTA final) and Tb-anti-pMAP2K1 (2 nM final). The plate was further incubated at room temperature for another 60 minutes, and the fluorescent signals were read on Victor 5 (Perkin Elmer) with excitation at 340 nM and emission at 495 and 520 nM. The assay signal was determined as a ratio of FRET-specific signal measured with emission filter at 520 nM to that of the signal measured with Tb-specific emission filter at 495 nM. IC₅₀ value was calculated using appropriate programs in GraphPad Prism by plotting the logarithm of the concentration versus percent inhibition. The IC₅₀ values for the example compounds are shown in Table 1.

Cell Proliferation Assay:

A375, Colo-205, Calu-6, and SW-480 cells were purchased from American Type Culture Collection (USA). All cells were cultured in the recommended medium and serum concentration. Cells were maintained at 37° C. in a humidified atmosphere with 5% CO₂. For cell proliferation assay, cells were seeded in 96-well pates at a density of 1,000 to 5,000 cells per well and cultured overnight at 37° C. in medium supplemented with 5-10% FBS. On the next day, the test articles at various concentrations or vehicle control (1% DMSO) were added into cell culture. After 3-day treatment, the growth of cells was assayed by the CellTiter-Glo® Luminestceaent Cell Viability Assay (Promega). IC₅₀ values were calculated using GraphPad Prism by plotting the logarithm of the concentration versus percent inhibition of cell growth as compared with vehicle control. The IC₅₀ values for the example compounds are shown in Table 1.

TABLE 1
Results from biological assays of the exemplified compounds

| | | BRAF^{V600E} | A375 cell |
|---|---|---|---|
| Example | | Lantha IC₅₀ | growth IC₅₀ |
| No. | Compound Name | (μM) | (μM) |
| 1 | N-[3-(3-amino-7-isoquinolyl)- | 8.69 | 0.103 |
| | 2,4-difluoro-phenyl]propane-1- | | |
| | sulfonamide | | |
| 2 | methyl N-[(1S)-2-[[7-[2,6- | 0.628 | 0.775 |
| | difluoro-3- | | |
| | (propylsulfonylamino)phenyl]- | | |
| | 3-isoquinolyl]amino]-1- | | |
| | methyl-ethyl]carbamate | | |
| 3 | N-[3-(3-amino-6-methoxy-7- | 0.799 | 1 |
| | isoquinolyl)-2,4-difluoro- | | |
| | phenyl]propane-1-sulfonamide | | |
| 4 | methyl N-[(1S)-2-[[7-[2,6- | 0.212 | 0.019 |
| | difluoro-3- | | |
| | (propylsulfonylamino)phenyl]- | | |
| | 6-methoxy-3- | | |
| | isoquinolyl]amino]-1-methyl- | | |
| | ethyl]carbamate | | |
| 5 | methyl N-[(1R)-2-[[7-[2,6- | 3.66 | 0.726 |
| | difluoro-3- | | |
| | (propylsulfonylamino)phenyl]- | | |
| | 6-methoxy-3- | | |
| | isoquinolyl]amino]-1-methyl- | | |
| | ethyl]carbamate | | |
| 6 | methyl N-[(1S)-2-[[7-[2,6- | 0.1 | 0.158 |
| | difluoro-3- | | |
| | (propylsulfonylamino)phenyl]- | | |
| | 6-(2-fluoroethoxy)-3- | | |
| | isoquinolyl]amino]-1-methyl- | | |
| | ethyl]carbamate | | |
| 7 | methyl N-[(1S)-2-[[7-[2,6- | 0.56 | 0.306 |
| | difluoro-3- | | |
| | (propylsulfonylamino)phenyl]- | | |
| | 6-ethyl-3-isoquinolyl]amino]- | | |
| | 1-methyl-ethyl]carbamate | | |
| 8 | N-[2,4-difiuoro-3-(3H- | 0.07 | 0.227 |
| | pyrazolo[3,4-c]isoquinolin-7- | | |
| | yl)phenyl]propane-1- | | |
| | sulfonamide | | |
| 9 | N-[3-(1-bromo-3H- | | 0.145 |
| | pyrazolo[3,4-c]isoquinolin-7- | | |
| | yl)-2,4-difluoro- | | |
| | phenyl]propane-1-sulfonamide | | |
| 10 | N-[3-(1-cyclopropyl-3H- | 2.71 | 0.145 |
| | pyrazolo[3,4-c]isoquinolin-7- | | |
| | yl)-2,4-difluoro- | | |
| | phenyl]propane-1-sulfonamide | | |
| 11 | N-[2-chloro-4-fluoro-3-(3H- | 0.0183 | 0.095 |
| | pyrazolo[3,4-c]isoquinolin-7- | | |
| | yl)phenyl]propane-1- | | |
| | sulfonamide | | |
| 12 | N-[3-(1-bromo-3H- | | 0.144 |
| | pyrazolo[3,4-c]isoquinolin-7- | | |
| | yl)-2-chloro-4-fluoro- | | |
| | phenyl]propane-1-sulfonamide | | |
| 13 | N- [2-chloro-3-(1-cyclopropyl- | 0.02 | 0.064 |
| | 3H-pyrazolo[3,4-c]isoquinolin- | | |
| | 7-yl)-4-fluoro-phenyl]propane- | | |
| | 1-sulfonamide | | |
| 14 | N-[2,4-difiuoro-3-(3H- | 0.05 | 0.066E |
| | pyrazolo[3,4-c]isoquinolin-7- | | |
| | yl)phenyl]-3-fluoro-propane-1- | | |
| | sulfonamide | | |
| 15 | N-[2-chloro-4-fluoro-3-(3H- | 0.0066 | 0.023 |
| | pyrazolo[3,4-c]isoquinolin-7- | | |
| | yl)phenyl]-3-fluoro-propane-1- | | |
| | sulfonamide | | |
| 16 | N-[2,4-dichloro-3-(3H- | 0.0413 | 0.341 |
| | pyrazolo[3,4-c]isoquinolin-7- | | |
| | yl)phenyl]propane-1- | | |
| | sulfonamide | | |
| 17 | N-[4-chloro-2-fluoro-3-(3H- | 0.118 | 0.131 |
| | pyrazolo[3,4-c]isoquinolin-7- | | |
| | yl)phenyl]propane-1- | | |
| | sulfonamide | | |
| 18 | 2-[2,4-difluoro-3-(3H- | 3.74 | 1 |
| | pyrazolo[3,4-c]isoquinolin-7- | | |
| | yl)phenyl]-1,2-thiazolidine | | |
| | 1,1-dioxide | | |
| 19 | N-[2,4-difluoro-3-(8-methoxy- | 0.06 | 0.077 |
| | 3H-pyrazolo[3,4-c]isoquinolin- | | |
| | 7-yl)phenyl]propane-1- | | |
| | sulfonamide | | |
| 20 | N-[2-chloro-4-fluoro-3-(8- | 0.01 | 0.047 |
| | methoxy-3H-pyrazolo[3,4- | | |
| | c]isoquinolin-7- | | |
| | yl)phenyl]propane-1- | | |
| | sulfonamide | | |
| 21 | N-[2,4-difluoro-3-(8-methoxy- | 0.07 | 0.094 |
| | 3H-pyrazolo[3,4-c]isoquinolin- | | |
| | 7-yl)phenyl]-3-fluoro-propane- | | |
| | 1-sulfonamide | | |
| 22 | N-[2-chloro-4-fluoro-3-(8- | 0.012 | 0.066 |
| | methoxy-3H-pyrazolo[3,4- | | |
| | c]isoquinolin-7-yl)phenyl]-3- | | |
| | fluoro-propane-1-sulfonamide | | |
| 23 | N-[3-(1-cyclopropyl-8- | 0.22 | 0.1 |
| | methoxy-3H-pyrazolo[3,4- | | |
| | c]isoquinolin-7-yl)-2,4- | | |
| | difluoro-phenyl]propane-1- | | |
| | sulfonamide | | |
| 24 | N-[2-chloro-3-(1-cyclopropyl- | 0.08 | 0.284 |
| | 8-methoxy-3H-pyrazolo[3,4- | | |
| | c]isoquinolin-7-yl)-4-fluoro- | | |
| | phenyl]propane-1-sulfonamide | | |
| 25 | N-[2-chloro-3-(1-cyclopropyl- | 0.007 | 0.025 |
| | 8-methoxy-3H-pyrazolo[3,4- | | |
| | c]isoquinolin-7-yl)-4-fluoro- | | |
| | phenyl]-3-fluoro-propane-1- | | |
| | sulfonamide | | |
| 26 | N-[3-(1-cyclopropyl-8- | 0.0488 | 0.0297 |
| | methoxy-3H-pyrazolo[3,4- | | |
| | c]isoquinolin-7-yl)-2,4- | | |
| | difluoro-phenyl]-3-fluoro- | | |
| | propane-1-sulfonamide | | |
| 27 | N-[2,4-difluoro-3-(3H- | 0.69 | 1 |
| | pyrrolo[2,3-c]isoquinolin-7- | | |
| | yl)phenyl]propane-1- | | |
| | sulfonamide | | |
| 28 | N-[2,4-difluoro-3-(3H- | 0.389 | 0.309 |
| | imidazo[4,5-c]isoquinolin-7- | | |
| | yl)phenyl]propane-1- | | |
| | sulfonamide | | |

### EXAMPLES

Certain preferred embodiments of the present invention are illustratively shown in the following non-limiting examples.

### Example 1

### N-(3-(3-aminoisoquinolin-7-yl)-2,4-difluorophenyl)propane-1-sulfonamide

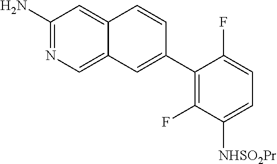

To a solution of sodium methoxide (65 mg) in dry methanol (6 mL) was added 2,2-diethoxyacetonitrile (1.29 g) at 0° C. The reaction mixture was stirred at room temperature for 2 hs. 3-bromo benzylic amine (1.49 g) was added. The reaction mixture was heated at 70° C. for 2 h and then concentrated to give crude acetimidamide under decreased pressure.

The above crude intermediate was dissolved in concentrated sulfuric acid (8 mL) and stirred at room temperature for 36 h. The reaction mixture was poured into iced water and basified to PH 9-10. After extracted with ethyl acetate (3×100 mL) and dried on Na₂SO₄, it was concentrated and purified on a silica gel column to give 7-bromoisoquinolin-3-amine of 1.3 g. ¹H NMR (400 MHz, CDCl₃): δ 8.79 (s, 1H), 7.93 (s, 1H), 7.55 (d, J=8.8 Hz, 1H), 7.42 (d, J=8.8 Hz, 1H), 6.70 (s, 1H); LC-MS: 223 (M+1).

7-Bromoisoquinolin-3-amine (150 mg), bis(pinacolato)diboron (203 mg), potassium acetate (218 mg) and Pd(dppf)Cl₂ (25 mg) were mixed into a microwave tube. Dioxane (4 ml) was added. The mixture was evacuated and flushed with nitrogen for three times. The reaction was carried out in microwave wave conditions at 120° C. for 1.5 h. The reaction mixture was cooled to room temperature and diluted with 100 mL ethyl acetate. After washed with water, brine and dried on Na₂SO₄, the reaction mixture was concentrated to give crude boronic ester. LC-MS: 271 (M+1).

The above crude boronic ester intermediate was mixed with N-(3-bromo-2,4-difluorophenyl)propane-1-sulfonamide (211 mg), sodium carbonate (235 mg) and Pd(dppf)Cl₂ (25 mg) in the solution of DME (4 mL) and water (0.5 mL). The mixture was evacuated and flushed with nitrogen for three times. The reaction was carried out in microwave wave conditions at 120° C. for 1.5 h. The reaction mixture was cooled to room temperature and diluted with 100 mL ethyl acetate. After washed with water, brine and dried on Na₂SO₄, it was concentrated and purified on a silica gel column to give desired product of 40 mg in 22% yields. ¹H NMR (400 MHz, CD₃OD): δ 8.83 (s, 1H), 7.92 (s, 1H), 7.64 (d, J=8.8 Hz, 1H), 7.53-7.57 (m, 2H), 7.11-7.14 (m, 1H), 6.84 (s, 1H), 3.10-3.14 (m, 2H), 1.85-1.91 (m, 2H), 1.06 (t, J=7.5 Hz, 3H). LC-MS: 378 (M+1).

### Example 2

### (S)-methyl-1-(7-(2,6-difluoro-3-(propylsulfonamido)phenyl)isoquinolin-3-ylamino)propan-2-ylcarbamate

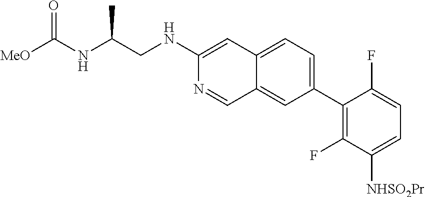

N-(3-(3-Aminoisoquinolin-7-yl)-2,4-difluorophenyl)propane-1-sulfonamide (22 mg) and (S)-methyl 1-oxopropan-2-ylcarbamate (12 mg) were dissolved in the solution of methanol (5 mL) and acetic acid (0.4 mL). The mixture was stirred for 20 mins, followed by addition of NaBH₃CN in one portion. The reaction mixture was stirred overnight under N₂ and then quenched with saturated NaHCO₃. After extracted with ethyl acetate, washed with water, brine and dried on Na₂SO₄, it was concentrated and purified on a silica gel column to give desired product of 12 mg in 42% yield. ¹H NMR (400 MHz, CDCl₃): δ 8.89 (s, 1H), 7.87 (s, 1H), 7.58-7.66 (m, 3H), 7.04-7.09 (m, 1H), 6.65 (s, 1H), 3.72-3.74 (m, 2H), 3.67 (s, 3H), 3.37-3.41 (m, 2H), 3.07-3.11 (m, 2H), 1.89-1.92 (m, 2H), 1.17 (d, J=6.8 Hz, 3H), 1.06 (t, J=7.5 Hz, 3H). LC-MS: 493 (M+1).

### Example 3

### N-(3-(3-amino-6-methoxyisoquinolin-7-yl)-2,4-difluorophenyl)propane-1-sulfonamide

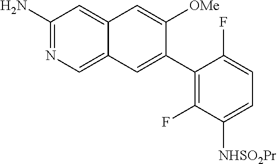

To a solution of sodium methoxide (25 mg) in dry methanol (8 mL) was added 2,2-diethoxyacetonitrile (0.5 g) at 0° C. The reaction mixture was stirred at room temperature for 2 hs. (3-bromo-4-methoxyphenyl)methanamine (0.24 g) was added. The reaction mixture was heated at 70° C. for 2 h and then concentrated to give crude acetimidamide under decreased pressure without further purification.

Above crude intermediate was dissolved in concentrated sulfuric acid (4 mL) and stirred at room temperature for 14 h. The reaction mixture was poured into iced water and basified to PH 9-10. After extracted with ethyl acetate (3×50 mL) and dried on Na₂SO₄, it was concentrated and purified on a silica gel column to give 7-bromo-6-methoxyisoquinolin-3-amine of 50 mg in 18% yield. ¹H NMR (400 MHz, CDCl₃)□: δ 8.65 (s, 1H), 7.96 (s, 1H), 6.80 (s, 1H), 6.61 (s, 1H), 3.98 (s, 3H); LC-MS: m/z 253.1 (M+H).

7-Bromo-6-methoxyisoquinolin-3-amine (50 mg), bis(pinacolato)diboron (61 mg), potassium acetate (65 mg) and Pd(dppf)Cl₂ (14.6 mg) was mixed into a microwave tube. Dioxane (3 ml) was added. The mixture was evacuated and flushed with nitrogen for three times. The reaction was carried out in microwave wave conditions at 120° C. for 1 h. The reaction mixture was cooled to room temperature and diluted with 20 mL ethyl acetate. After washed with water, brine and dried on Na₂SO₄, it was concentrated to give crude boronic ester. LC-MS: 301.2 (M+H).

Above crude boronic ester intermediate (30 mg) was mixed with N-(3-bromo-2,4-difluorophenyl)propane-1-sulfonamide (37 mg), Cs₂CO₃ (97 mg) and Pd(dppf)Cl₂ (7.3 mg) in the solution of DMF (3 mL) and water (0.3 mL). The mixture was evacuated and flushed with nitrogen for three times. The reaction was carried out in microwave wave conditions at 120° C. for 1 h. The reaction mixture was cooled to room temperature and diluted with 50 mL ethyl acetate. After washed with water, brine and dried on Na₂SO₄, it was concentrated and purified on a silica gel column to give desired product of 19 mg in 47% yields. ¹HNMR (400 MHz, CDCl₃): □ δ 8.69 (s, 1H), 7.63 (s, 1H), 7.57-7.60 (m, 1H), 6.97-6.99 (m, 1H), 6.87 (s, 1H), 6.66 (s, 1H), 4.75 (s, br, 2H), 3.87 (s, 3H), 3.04-3.09 (m, 2H), 1.86-1.92 (m, 2H), 1.02 (t, J=7.5 Hz, 3H); LC-MS: 408.1 (M+H).

### Example 4

### N—((S)-1-(7-(2,6-difluoro-3-(propylsulfonamido)phenyl)-6-meyhoxyisoquinolin-3-ylamino)propan-2-yl)acetamide

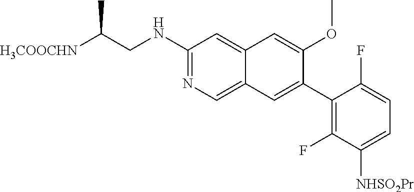

To a solution of N-(3-(3-amino-6-methoxyisoquinolin-7-yl)-2,4-difluorophenyl)propane-1-sulfonamide (12 mg) and (S)-methyl 1-oxopropan-2-ylcarbamate (5.8 mg) in CH₃OH (2 ml) was added acetic acid (50 mg). The mixture was stirred at room temperature for 0.5 h, then NaBH₃CN (3.78 mg) was added. The above mixture was stirred for another 10 hrs. Saturated NaHCO₃ (10 mL) was added to it, followed by ethyl acetate (20 mL). After washed with water, brine and dried on Na₂SO₄, it was concentrated and purified on a silica gel column to give desired product of 3.0 mg in 20% yields. ¹H NMR (400 MHz, CDCl₃)□ δ 8.67 (s, 1H), 7.60 (s, 1H), 7.57-7.60 (m, 1H), 6.97-6.99 (m, 1H), 6.92 (s, 1H), 6.57 (s, 1H), 3.88 (s, 3H), 3.55 (s, 3H) 3.49-3.51 (m, 2H), 3.07-3.09 (m, 2H), 1.87-1.93 (m, 2H), 1.29-1.31 (m, 3H), 1.06 (t, J=7.50, 3H); LC-MS: 523.2 (M+H).

### Example 5

### N—((R)-1-(7-(2,6-difluoro-3-(propylsulfonamido)phenyl)-6-meyhoxyisoquinolin-3-ylamino)propan-2-yl)acetamide

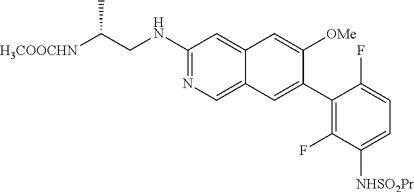

The above similar procedures of analogues A were followed to give the product with (R)-methyl 1-oxopropan-2-ylcarbamate as starting material. LC-MS: 523.2 (M+H).

### Example 6

### N—((S)-1-(7-(2,6-difluoro-3-(propylsulfonamido)phenyl)-6-(2-fluoroethoxy)isoquinolin-3-ylamino)propan-2-yl)acetamide

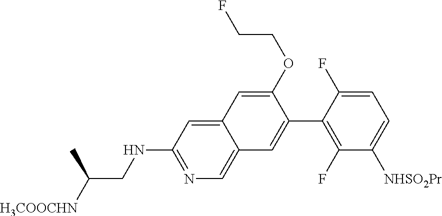

The above similar procedures of analogues A were followed to give the product. ¹HNMR (400 MHz, CDCl₃)□ δ 8.67 (s, 1H), 7.66 (s, 1H), 7.59-7.62 (m, 1H), 6.88-6.92 (m, 1H), 6.78 (s, 1H), 6.50 (s, 1H), 4.86-4.88 (m, 1H), 4.72-4.76 (m, 1H), 4.33-4.35 (m, 1H), 4.26-4.28 (m, 1H), 3.72 (s, 3H) 3.49-3.51 (m, 2H), 3.07-3.09 (m, 2H), 1.89-1.92 (m, 2H), 1.28-1.30 (m, 3H), 1.04 (t, J=7.52, 3H); LC-MS: 555.2 (M+H).

### Example 7

### N—((S)-1-(7-(2,6-difluoro-3-(propylsulfonamido)phenyl)-6-ethylisoquinolin-3-ylamino)propan-2-yl)acetamide

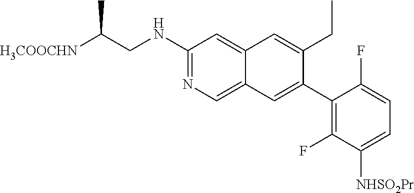

The above similar procedures of analogues A were followed to give the product. LC-MS: 521.2.1 (M+H).

### Synthesis of Analogues B

### Example 8

### N-(2,4-difluoro-3-(3H-pyrazolo[3,4-c]isoquinolin-7-yl)phenyl)propane-1-sulfonamide

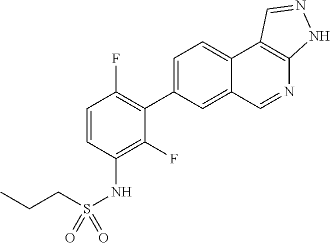 [SMILES: CCCS(=O)(=O)Nc1ccc(F)c(-c2ccc3c(cnc4[nH]ncc43)c2)c1F]

To a mixture of POCl₃ (35 g, 0.228 mol) and DMF (16 g, 0.228 mol) in THF (100 mL) 7-bromo-1,2-dihydroisoquinolin-3(4H)-one (20 g, 0.088 mol) was added at 0° C. in portions during 40 min. The mixture was stirred at 0° C. for 3 hours and then poured into ice. It was neutralized to PH=7 with 2N NaOH, extracted with DCM. The combined organic layer was dried with Na₂SO₄ and concentrated to provide crude (7-bromo-3-chloroisoquinolin-4(1H)-ylidene)-N,N-dimethylmethanamine of 26 g as red oil.

To above crude intermediate in toluene (150 mL) was added 2N H₂SO₄ (150 mL) under vigorous stirred and then KMnO₄ (12 g) was added in portions at room temperature. The mixture was stirred for another 6 hours, filtered and the organic phase was separated, dried and evaporated. The residue was crystallized from ethyl acetate to give 7-bromo-3-chloroisoquinoline-4-carbaldehyde of 4 g in 17% overall yields as yellow solid. ¹H NMR (400 MHz, DMSO-d6)□: δ 10.65 (s, 1H), 9.43 (s, 1H), 8.84 (d, J=9.5 Hz, 1H), 8.61 (s, 1H), 8.14 (d, J=9.1 Hz, 1H); LC-MS: 270 (M+1).

Hydrazine (20 mL) was added over 5 min to a solution of 7-bromo-3-chloroisoquinoline-4-carbaldehyde (4 g, 0.015 mol) in DME (20 mL). The reaction mixture was refluxed overnight and concentrated in vacuo. Water was added to the mixture. The resulting precipitate was filtered off. The solid was added in Hydrazine (20 mL), the mixture was heated at 100° C. overnight. Water was added to the mixture. The resulting precipitate was filtered off to provide 7-bromo-3H-pyrazolo[3,4-c]isoquinoline of 2 g in 54% yield as yellow solid. ¹HNMR (400 MHz, DMSO-d6)□: δ 9.15 (s, 1H), 8.63 (s, 1H), 8.52 (s, 1H), 8.33 (d, J=9.3 Hz, 1H), 8.03 (d, J=9.1 Hz, 1H); LC-MS: 250 (M+1).

7-Bromo-3H-pyrazolo[3,4-c]isoquinoline (2 g, 8.06 mmol) was dissolved in dioxane (20 mL), then potassium acetate (2.37 g, 24.19 mmol), Pd(dppf)Cl₂ (295 mg, 0.40 mmol) and 4,4,5,5-tetramethyl-2-(4,4,5,5-tetramethyl-1,3,2-dioxaborolan-2-yl)-1,3,2-dioxaborolane (2.66 g, 10.48 mmol) were added. The reaction mixture was evacuated and flushed with nitrogen for three times and stirred at 100° C. overnight. After cooled, filtered and washed with ethyl acetate, the filtrate was washed with brine, dried on Na₂SO₄ and concentrated. The residue was purified by flash chromatography to give 7-(4,4,5,5-tetramethyl-1,3,2-dioxaborolan-2-yl)-3H-pyrazolo[3,4-c]isoquinoline of 1.5 g in 65% yield as yellow solid. ¹HNMR (400 MHz, CDCl₃): δ 11.56 (br, 1H), 9.14 (s, 1H), 8.62 (s, 1H), 8.48 (s, 1H), 8.19-8.24 (m, 2H), 1.42 (s, 9H); LC-MS: 295 (M+1).

To a solution of 7-(4,4,5,5-tetramethyl-1,3,2-dioxaborolan-2-yl)-3H-pyrazolo[3,4-c]isoquinoline (1.5 g, 5.085 mmol) in DCM (20 mL) was added triethylamine (0.77 g, 7.627 mmol) and acetic anhydride (0.78 g, 7.627 mmol). The reaction mixture was stirred at room temperature overnight. After diluted with DCM and washed with brine, the organic layer was dried with Na₂SO₄ and concentrated to provide 1-(7-(4,4,5,5-tetramethyl-1,3,2-dioxaborolan-2-yl)-3H-pyrazolo[3,4-c]isoquinolin-3-yl)ethanone of 1.6 g in 93% yield as yellow solid. LC-MS: 338 (M+1). To a solution of 1-(7-(4,4,5,5-tetramethyl-1,3,2-dioxaborolan-2-yl)-3H-pyrazolo[3,4-c]isoquinolin-3-yl)ethanone (50 mg, 0.148 mmol) in DMF (2.5 mL) was added N-(3-bromo-2,4-difluorophenyl)propane-1-sulfonamide (46 mg, 0.148 mmol), 2M Na₂CO₃ (0.3 mmol, 0.3 mL) and Pd(dppf)Cl₂ (8 mg). The reaction mixture was evacuated and flushed with nitrogen for three times and stirred under microwave at 150° C. for 1.5 hours. The mixture was diluted with water and extracted with ethyl acetate. The organic layer was washed with brine, dried over Na₂SO₄ and concentrated. The residue was purified by PTLC to give N-(2,4-difluoro-3-(3H-pyrazolo[3,4-c]isoquinolin-7-yl)phenyl) propane-1-sulfonamide of 11 mg in 18% yield as white solid. ¹HNMR (400 MHz, DMSO-d6):□ δ 14.02 (s, 1H), 9.73 (s, 1H), 9.24 (s, 1H), 8.66 (s, 1H), 8.50 (d, J=8.5 Hz, 1H), 8.37 (s, 1H), 7.95 (d, J=8.2 Hz, 1H), 7.50 (t, J=3.2 Hz, 1H), 7.30 (t, J=9.1 Hz, 1H), 3.11-3.15 (m, 2H), 1.74-1.79 (m, 2H), 0.85 (t, J=7.5 Hz, 3H); LC-MS: 402 (M+1).

### Example 9

### N-(3-(1-bromo-3H-pyrazolo[3,4-c]isoquinolin-7-yl)-2,4-difluorophenyl)propane-1-sulfonamide

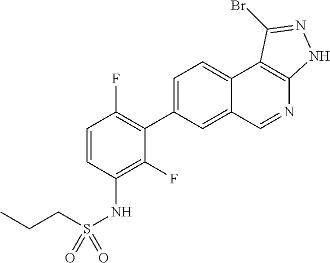 [SMILES: CCCS(=O)(=O)Nc1ccc(F)c(-c2ccc3c(cnc4[nH]nc(Br)c43)c2)c1F]

A mixture of N-(2,4-difluoro-3-(3H-pyrazolo[3,4-c]isoquinolin-7-yl)phenyl)propane-1-sulfonamide (100 mg, 0.25 mmol), NBS (46 mg, 0.26 mmol) in CH₃CN (15 mL) was stirred at room temperature for 3 hours. After removing solvent in vacuum, the residue was diluted with water and extracted with DCM. The organic layer was washed with brine, dried over Na₂SO₄ and concentrated, the residue was purified by column chromatography to give N-(3-(1-bromo-3H-pyrazolo[3,4-c]isoquinolin-7-yl)-2,4-difluorophenyl)propane-1-sulfonamide of 80 mg in 66% yield as yellow solid. ¹HNMR (400 MHz, DMSO-d6)□: δ 14.46 (s, 1H), 9.74 (s, 1H), 9.32 (s, 1H), 8.84 (d, J=8.5 Hz, 1H), 8.45 (s, 1H), 8.08 (d, J=8.0 Hz, 1H), 7.52 (t, J=3.1 Hz, 1H), 7.31 (t, J=9.1 Hz, 1H), 3.11-3.15 (m, 2H) 1.75-1.77 (m, 2H), 0.98 (t, J=7.2 Hz, 3H); LC-MS: 481 (M+1).

### Example 10

### N-(3-(1-cyclopropyl-3H-pyrazolo[3,4-c]isoquinolin-7-yl)-2,4-difluorophenyl)propane-1-sulfonamide

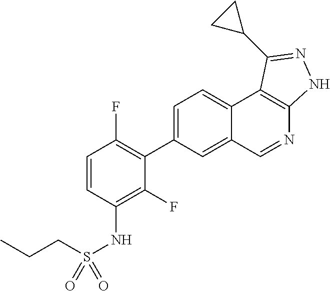 [SMILES: CCCS(=O)(=O)Nc1ccc(F)c(-c2ccc3c(cnc4[nH]nc(C5CC5)c43)c2)c1F]

A solution of N-(3-(1-bromo-3H-pyrazolo[3,4-c]isoquinolin-7-yl)-2,4-difluorophenyl)propane-1-sulfonamide (80 mg, 0.166 mmol) in DCM (20 mL) was treated with triethyamine (25 mg, 0.249 mmol), DMAP (5 mg) and Di-tert-butyl dicarbonate (54 mg, 0.249 mmol). The mixture was stirred at room temperature for 5 hours. After diluted with DCM and washed with brine, the organic layer was dried with Na₂SO₄ and concentrated, the residue was purified by PTLC to give tert-butyl 1-bromo-7-(2,6-difluoro-3-(propylsulfonamido)phenyl)-3H-pyrazolo[3,4-c]isoquinoline-3-carboxylate of 30 mg in 31% yield. LC-MS: 581 (M+1).

To a reaction vessel was added tert-butyl 1-bromo-7-(2,6-difluoro-3-(propylsulfonamido)phenyl)-3H-pyrazolo[3,4-c]isoquinoline-3-carboxylate (30 mg, 0.051 mmol), cyclopropylboronic acid (9 mg, 0.103 mmol) and potassium phosphate (33 mg, 0.155 mmol). Toluene (3 mL) and water (0.3 mL) was added. The mixture was purged with nitrogen for 10 min, and then palladium (II) acetate (2 mg) and tri-cyclohexyl phosphine (2 mg) were added. The reaction vessel was purged with nitrogen and sealed. The reaction mixture was stirred under microwave at 150° C. for 1.5 hours. After cooled to room temperature and diluted with water and extracted with EA, the organic layer was washed with brine, dried over Na₂SO₄ and concentrated. The residue was purified by PTLC to give tert-butyl 1-cyclopropyl-7-(2,6-difluoro-3-(propylsulfonamido)phenyl)-3H-pyrazolo[3,4-c]isoquinoline-3-carboxylate of 15 mg in 53% yield. LC-MS: 543 (M+1).

To a solution of tert-butyl 1-cyclopropyl-7-(2,6-difluoro-3-(propylsulfonamido)phenyl)-3H-pyrazolo[3,4-c]isoquinoline-3-carboxylate (15 mg, 0.027 mmol) in DCM (2 mL) was added TFA (2 mL). The mixture was stirred at room temperature for 3 hours. After removing the solvent in vacuum, the residue was partitioned between ethyl acetate and saturated sodium bicarbonate, washed with brine. The organic layer was dried on Na₂SO₄ and concentrated. The residue was purified by PTLC to give N-(3-(1-cyclopropyl-3H-pyrazolo[3,4-c]isoquinolin-7-yl)-2,4-difluorophenyl)propane-1-sulfonamide of 4 mg in 33% yield as white solid. ¹HNMR (400 MHz, DMSO-d6)□: δ 13.59 (s, 1H), 9.74 (s, 1H), 9.20 (s, 1H), 8.67 (d, J=8.4 Hz, 1H), 8.37 (s, 1H), 7.97 (d, J=8.5 Hz, 1H) 7.47-7.53 (m, 1H), 7.29 (t, J=8.8 Hz, 1H), 3.11-3.15 (m, 2H), 1.98-2.02 (m, 1H), 1.75-1.77 (m, 2H), 1.11-1.20 (m, 2H), 0.97-1.00 (m, 5H); LC-MS: 443 (M+1).

### Example 11

### N-(2-chloro-4-fluoro-3-(3H-pyrazolo[3,4-c]isoquinolin-7-yl)phenyl)propane-1-sulfonamide

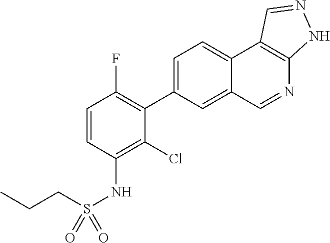 [SMILES: CCCS(=O)(=O)Nc1ccc(F)c(-c2ccc3c(cnc4[nH]ncc43)c2)c1Cl]

To a solution of 3-bromo-2-chloro-4-fluorobenzenamine (200 mg, 0.89 mmol), triethylamine (361 mg, 3.57 mmol) in DCM (10 mL), was treated with n-propanesulfonyl chloride (318 mg, 2.23 mmol). The mixture was stirred at room temperature overnight. Aqueous sodium bicarbonate was added, and the mixture was extracted with DCM. The combined organic layer were dried on Na₂SO₄ and concentrated. The residue was dissolved in acetonitrile (10 mL) and aqueous sodium carbonate solution (375 mg, 3.57 mmol) was added. The reaction mixture was refluxed for 2 hours, cooled to room temperature and then extracted with ethyl acetate, washed wished with brine, and concentrated. The residue was purified by column chromatography on silica gel to give N-(3-bromo-2-chloro-4-fluorophenyl)propane-1-sulfonamide of 180 mg in 61% yield as white solid. ¹HNMR (400 MHz, CDCl₃)□: δ 7.66-7.70 (m, 1H), 7.10-7.14 (m, 1H), 6.37 (br_s, 1H), 3.03-3.07 (m, 2H), 1.84-2.05 (m, 2H), 1.05 (t, J=7.5 Hz, 3H); LC-MS: 330 (M+1).

To a solution of 1-(7-(4,4,5,5-tetramethyl-1,3,2-dioxaborolan-2-yl)-3H-pyrazolo[3,4-c]isoquinolin-3-yl)ethanone (50 mg, 0.148 mmol) in DMF (2.5 mL) was added N-(3-bromo-2-chloro-4-fluorophenyl)propane-1-sulfonamide (49 mg, 0.148 mmol), 2M Na₂CO₃ (0.3 mmol, 0.3 mL) and Pd(dppf)Cl₂ (8 mg). The reaction mixture was stirred under microwave at 150° C. for 1.5 hours. The mixture was diluted with water and extracted with ethyl acetate. The organic layer was washed with brine, dried over Na₂SO₄ and concentrated. The residue was purified by PTLC to give N-(2-chloro-4-fluoro-3-(3H-pyrazolo[3,4-c]isoquinolin-7-yl)phenyl)propane-1-sulfonamide of 6 mg in 9% yield as white solid. ¹HNMR (400 MHz, DMSO-d6)□: δ 14.01 (s, 1H), 9.62 (s, 1H), 9.21 (s, 1H), 8.65 (s, 1H), 8.48 (d, J=8.5 Hz, 1H), 8.27 (s, 1H), 7.86 (d, J=8.2 Hz, 1H), 7.59 (t, J=3.1 Hz, 1H), 7.45 (t, J=9.0 Hz, 1H), 3.13-3.16 (m, 2H), 1.75-1.79 (m, 2H), 0.98 (t, J=7.2 Hz, 3H); LC-MS: 419 (M+1).

### Example 12

### N-(3-(1-bromo-3H-pyrazolo[3,4-c]isoquinolin-7-yl)-2-chloro-4-fluorophenyl)propane-1-sulfonamide

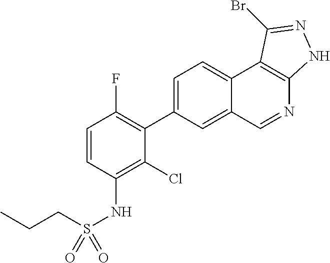 [SMILES: CCCS(=O)(=O)Nc1ccc(F)c(-c2ccc3c(cnc4[nH]nc(Br)c43)c2)c1Cl]

A mixture of N-(2-chloro-4-fluoro-3-(3H-pyrazolo[3,4-c]isoquinolin-7-yl)phenyl)propane-1-sulfonamide (100 mg, 0.248 mmol), NBS (46 mg, 0.261 mmol) in CH₃CN (15 mL) was stirred at room temperature for 3 hours. The solvent was removed in vacuum. The residue was diluted with water and extracted with DCM. The organic layer was washed with brine, dried over Na₂SO₄ and concentrated. The residue was purified by column chromatography on silica gel to give N-(3-(1-bromo-3H-pyrazolo[3,4-c]isoquinolin-7-yl)-2-chloro-4-fluorophenyl) propane-1-sulfonamide of 60 mg in 50% yield as yellow solid. ¹HNMR (400 MHz, DMSO-d6)□: δ14.46 (s, 1H), 9.74 (s, 1H), 9.32 (s, 1H) 8.84 (d, J=8.5 Hz, 1H), 8.45 (s, 1H), 8.08 (d, J=8.0 Hz, 1H), 7.52 (t, J=3.1 Hz, 1H), 7.31 (t, J=9.1 Hz, 1H), 3.11-3.15 (m, 2H), 1.75-1.77 (m, 2H), 0.98 (t, J=7.2 Hz, 3H); LC-MS: 497 (M+1).

### Example 13

### N-(2-chloro-3-(1-cyclopropyl-3H-pyrazolo[3,4-c]isoquinolin-7-yl)-4-fluorophenyl)propane-1-sulfonamide

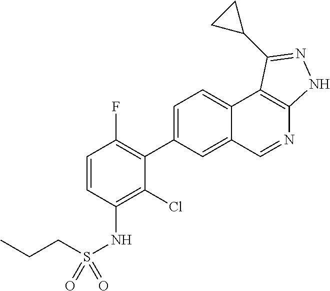 [SMILES: CCCS(=O)(=O)Nc1ccc(F)c(-c2ccc3c(cnc4[nH]nc(C5CC5)c43)c2)c1Cl]

A solution of N-(3-(1-bromo-3H-pyrazolo[3,4-c]isoquinolin-7-yl)-2-chloro-4-fluorophenyl)propane-1-sulfonamide (60 mg, 0.120 mmol) in DCM (20 mL) was treated with triethyamine (18 mg, 0.181 mmol), DMAP (5 mg) and Di-tert-butyl dicarbonate (39 mg, 0.181 mmol). The mixture was stirred at room temperature for 5 hours and then diluted with DCM, washed with brine. The organic layer was dried on Na₂SO₄ and concentrated. The residue was purified by PTLC to give tert-butyl 1-bromo-7-(2-chloro-6-fluoro-3-(propylsulfonamido)phenyl)-3H-pyrazolo[3,4-c]isoquinoline-3-carboxylate of 25 mg in 35% yield. LCMS: 598 (M+1).

To a reaction vessel was added tert-butyl 1-bromo-7-(2-chloro-6-fluoro-3-(propylsulfonamido)phenyl)-3H-pyrazolo[3,4-c]isoquinoline-3-carboxylate (20 mg, 0.033 mmol), cyclopropylboronic acid (6 mg, 0.066 mmol) and potassium phosphate (21 mg, 0.099 mmol). Toluene (3 mL) and water (0.3 mL) were added. The mixture was purged with nitrogen for 10 min. Palladium (II) acetate (2 mg) and tri-cyclohexyl phosphine (2 mg) was added. The reaction vessel was purged with argon and sealed. The mixture was stirred under microwave at 150° C. for 1.5 hours. The mixture was diluted with water and extracted with ethyl acetate. After the organic layer was washed with brine, dried over Na₂SO₄ and concentrated, the residue was purified by PTLC to give tert-butyl7-(2-chloro-6-fluoro-3-(propylsulfonamido)phenyl)-1-cyclopropyl-3H-pyrazolo[3,4-c]isoquinoline-3-carboxylate of 10 mg in 53% yield as colorless oil. LCMS: 560 (M+1).

To a solution of tert-butyl 7-(2-chloro-6-fluoro-3-(propylsulfonamido)phenyl)-1-cyclopropyl-3H-pyrazolo[3,4-c]isoquinoline-3-carboxylate (10 mg, 0.017 mmol) in DCM (2 mL) was added TFA (2 mL). The mixture was stirred at room temperature for 3 hours. The solvent was removed in vacuum, the residue partitioned between ethyl acetate and saturated sodium bicarbonate, washed with brine, the organic layer was dried Na₂SO₄ and concentrated. the residue was purified by PTLC to give N-(2-chloro-3-(1-cyclopropyl-3H-pyrazolo[3,4-c]isoquinolin-7-yl)-4-fluorophenyl)propane-1-sulfonamide of 2 mg in 24% yield as white solid. LC-MS: 459 (M+1).

### Example 14

### N-(2,4-difluoro-3-(3H-pyrazolo[3,4-c]isoquinolin-7-yl)phenyl)-3-fluoropropane-1-sulfonamide

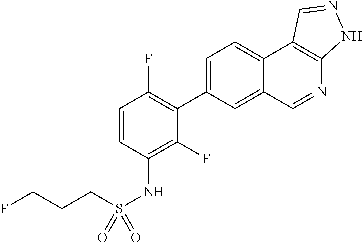 [SMILES: O=S(=O)(CCCF)Nc1ccc(F)c(-c2ccc3c(cnc4[nH]ncc43)c2)c1F]

The similar procedures of analogues B were followed to give the product. LC-MS: 421 (M+1).

### Example 15

### N-(2-chloro-4-fluoro-3-(3H-pyrazolo[3,4-c]isoquinolin-7-yl)phenyl)-3-fluoropropane-1-sulfonamide

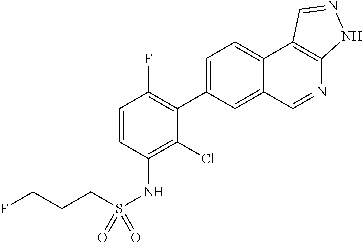 [SMILES: O=S(=O)(CCCF)Nc1ccc(F)c(-c2ccc3c(cnc4[nH]ncc43)c2)c1Cl]

The similar procedures of analogues B were followed to give the product. LC-MS: 437 (M+1).

### Example 16

### N-(2,4-dichloro-3-(3H-pyrazolo[3,4-c]isoquinolin-7-yl)phenyl)propane-1-sulfonamide

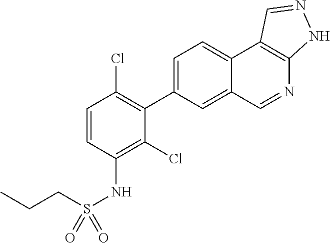 [SMILES: CCCS(=O)(=O)Nc1ccc(Cl)c(-c2ccc3c(cnc4[nH]ncc43)c2)c1Cl]

The similar procedures of analogues B were followed to give the product. ¹HNMR (400 MHz, CDCl₃) δ 9.18 (s, 1H), 8.53 (s, 1H), 8.35 (d, J=8.3 Hz, 1H), 8.03 (s, 1H), 7.72-7.77 (m, 2H), 7.50 (d, J=8.3 Hz, 1H), 7.01 (br, 1H), 3.08-3.11 (m, 2H), 1.90-1.95 (m, 2H), 1.09 (t, J=7.2 Hz, 3H). LC-MS: 435 (M+1).

### Example 17

### N-(4-chloro-2-fluoro-3-(3H-pyrazolo[3,4-c]isoquinolin-7-yl)phenyl)propane-1-sulfonamide

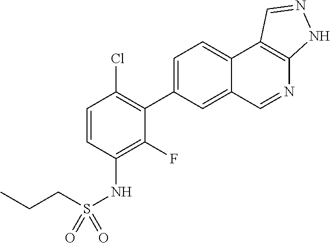 [SMILES: CCCS(=O)(=O)Nc1ccc(Cl)c(-c2ccc3c(cnc4[nH]ncc43)c2)c1F]

The similar procedures of analogues B were followed to give the product. ¹HNMR (400 MHz, DMSO-d6)□: δ 14.01 (s, 1H), 9.88 (s, 1H), 9.23 (s, 1H), 8.66 (s, 1H), 8.50 (d, J=8.3 Hz, 1H), 8.29 (s, 1H), 7.95 (s, 1H), 7.89 (d, J=8.2 Hz, 1H), 7.50-7.53 (m, 2H), 3.15-3.19 (m, 2H), 1.74-1.77 (m, 2H), 0.98 (t, J=7.6 Hz, 3H). LC-MS: 419 (M+1).

### Example 18

### N-(2,4-difluoro-3-(3H-pyrazolo[3,4-c]isoquinolin-7-yl)phenyl)-1,3-propanesultam

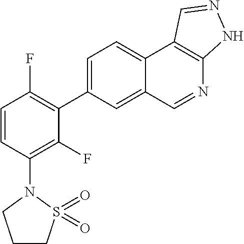 [SMILES: O=S1(=O)CCCN1c1ccc(F)c(-c2ccc3c(cnc4[nH]ncc43)c2)c1F]

The similar procedures of analogues B were followed to give the product. LC-MS: 401 (M+1).

### Synthesis of Analogues C

### Example 19

### N-(2,4-difluoro-3-(8-methoxy-3H-pyrazolo[3,4-c]isoquinolin-7-yl)phenyl)propane-1-sulfonamide

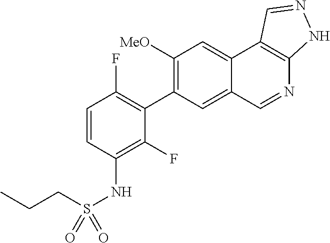 [SMILES: CCCS(=O)(=O)Nc1ccc(F)c(-c2cc3cnc4[nH]ncc4c3cc2OC)c1F]

To (4-bromo-3-methoxyphenyl) methanol (586 mg) in dry ether (8 mL) was added tribromo-phosphine (366 mg) at ° C. The reaction mixture was stirred for 2-3 hs while warming to room temperature slowly. It was poured into iced water and basified to PH 7. After extracted with ether (3×50 mL) and dried on Na₂SO₄, it was concentrated at below 30° C. to give crude 1-bromo-4-(bromomethyl)-2-methoxybenzene without further purification.

Above crude 1-bromo-4-(bromomethyl)-2-methoxybenzene was dissolved in DMSO (8 mL). Catalytic 18-Crown-6 and powder potassium cyanide (263 mg) was added at ° C. The reaction mixture was stirred at room temperature for 2 h. Water (20 mL) was added. After extracted with ether (3×50 mL), washed with brine for three times and dried on Na₂SO₄, it was concentrated to give the product of 2-(4-bromo-3-methoxyphenyl)acetonitrile of 533 mg in 91% yield. ¹HNMR (400 MHz, CDCl₃): δ 7.53 (d, J=8.3 Hz, 1H), 6.86 (d, J=1.5 Hz, 1H), 6.79-6.81 (m, 1H), 3.93 (s, 3H), 3.73 (s, 2H). LC-MS: 224 (M−1).

NaH (142 mg, 60% in mineral oil) was suspended in dry THF (10 mL) at 0° C., followed by the addition of the solution of 2-(4-bromo-3-methoxyphenyl)acetonitrile (533 mg) and methyl formic ester (213 mg) in dry THF (2 mL). The reaction mixture was stirred for 0.5 h at 0° C. and then heated at 50° C. for 0.5-1 h. Amounts of red solid was shown. It was cooled to room temperature and poured into iced water and acidified to PH 4-5. After extracted with ether (3×50 mL) and dried on Na₂SO₄, it was concentrated to give crude 2-(4-bromo-3-methoxyphenyl)-3-oxopropanenitrile of 541 mg as yellow solid without further purification. LC-MS: 252 (M−1).

Above crude nitrile (541 mg) and benzyl hydzine hydrochloric salt (373 mg) was dissolved in ⁱPrOH (5 mL) and acetic acid (0.29 mL). The reaction mixture was refluxed for 2-4 hs. It was cooled to room temperature and poured into iced water and neutralized with sodium bicarbonate. After extracted with ethyl acetate (3×50 mL) and dried on Na₂SO₄, it was concentrated to give crude 1-benzyl-4-(4-bromo-3-methoxyphenyl)-1H-pyrazol-5-amine of 746 mg as yellow solid without further purification. LC-MS: 360 (M+2).

1-Benzyl-4-(4-bromo-3-methoxyphenyl)-1H-pyrazol-5-amine (161 mg) and paraformaldehyde (14.8 mg) was dissolved in TFA (5 mL). The mixture was refluxed for 5-6 hrs. It was cooled to room temperature and poured into iced water and neutralized with sodium bicarbonate. After extracted with ethyl acetate (3×50 mL) and dried on Na₂SO₄ and concentrated, purified on a silica gel column to give 3-benzyl-7-bromo-8-methoxy-3H-pyrazolo[3,4-c]isoquinoline of 70 mg in 43% yield. ¹HNMR (400 MHz, CDCl₃): δ 8.87 (s, 1H), 8.36 (s, 1H), 8.27 (s, 1H), 7.45 (s, 1H), 7.35-7.38 (m, 2H), 7.29-7.31 (m, 3H), 5.80 (s, 2H), 4.11 (s, 3H); LC-MS: 369 (M+1).

3-Benzyl-7-bromo-8-methoxy-3H-pyrazolo[3,4-c]isoquinoline (63 mg), bis(pinacolato)diboron (52 mg), potassium acetate (50 mg) and Pd(dppf)Cl₂ (6.3 mg) was dissolved in dioxane (5 ml). The mixture was evacuated and flushed with nitrogen for three times and then was heated at 100° C. overnight. It was cooled to room temperature and diluted with 100 mL ethyl acetate. After washed with water, brine and dried on Na₂SO₄, it was concentrated to give crude boronic ester. LC-MS: 416 (M+1).

Above crude boronic ester was mixed with N-(3-bromo-2,4-difluorophenyl)propane-1-sulfonamide (53 mg), tripotassium phosphate (75 mg) and Bis[di-tert-butyl-(4-dimethylaminophenyl)phosphine]dichloropalladium (2.0 mg) in the solution of dioxane (5 mL) and water (0.4 mL). The reaction mixture was evacuated and flushed with nitrogen for three times and then was heated at 110° C. for 5 h. It was cooled to room temperature and diluted with 100 mL ethyl acetate. After washed with water, brine and dried on Na₂SO₄ and concentrated. The residue was purified on a silica gel column to give N-(3-(3-benzyl-8-methoxy-3H-pyrazolo[3,4-c]isoquinolin-7-yl)-2,4-difluorophenyl)propane-1-sulfonamide of 44 mg in 50% overall yields. ¹HNMR (400 MHz, CDCl₃): δ 8.97 (s, 1H), 8.41 (s, 1H), 7.98 (s, 1H), 7.62-7.63 (m, 1H), 7.57 (s, 1H), 7.28-7.36 (m, 5H), 7.04-7.07 (m, 1H), 6.59 (brs, 1H), 5.83 (s, 2H), 4.01 (s, 3H), 3.09-3.13 (m, 2H), 1.87-1.92 (m, 2H), 1.07 (t, J=7.5 Hz, 3H); LC-MS: 523 (M+1).

To a solution of N-(3-(3-benzyl-8-methoxy-3H-pyrazolo[3,4-c]isoquinolin-7-yl)-2,4-difluorophenyl)propane-1-sulfonamide (80 mg) in formic acid (8 mL) was added formic ammonium (38 mg) and Pd(OH)₂ (150 mg, 20% on Carbon). The reaction mixture was heated at 100° C. overnight. It was cooled to room temperature and filtered through a pad of Celite, washed with ethyl ester. The filtrate was concentrated to remove formic acid. The residue was re-dissolved in ethyl acetate (100 mL) and washed with saturated sodium bicarbonate. The organic layer was dried on Na₂SO₄ and concentrated. The residue was purified on a silica gel column to give N-(2,4-difluoro-3-(8-methoxy-3H-pyrazolo[3,4-c]isoquinolin-7-yl)phenyl)propane-1-sulfonamide of 27 mg in 41% yield. ¹HNMR (400 MHz, CD₃OD)□: δ 9.00 (s, 1H), 8.60 (s, 1H), 8.12 (s, 1H), 7.90 (s, 1H), 7.54-7.60 (m, 1H), 7.08-7.12 (m, 1H), 4.09 (s, 3H), 3.02-3.12 (m, 2H), 1.85-1.88 (m, 2H), 1.06 (t, J=7.2 Hz, 3H); LC-MS: 433 (M+1).

### Example 20

### N-(2-chloro-4-fluoro-3-(8-methoxy-3H-pyrazolo[3,4-c]isoquinolin-7-yl)phenyl)propane-1-sulfonamide

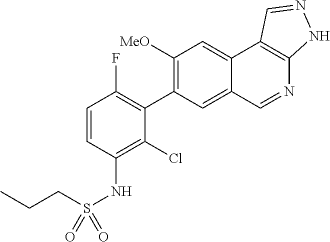 [SMILES: CCCS(=O)(=O)Nc1ccc(F)c(-c2cc3cnc4[nH]ncc4c3cc2OC)c1Cl]

The similar procedures of analogues C were followed to give the product. ¹HNMR (400 MHz, DMSO-d6)□: δ 13.85 (s, 1H), 9.56 (s, 1H), 9.04 (s, 1H), 8.64 (s, 1H), 8.12 (s, 1H), 7.98 (s, 1H), 7.54-7.58 (m, 1H), 7.36-7.40 (m, 1H), 4.02 (s, 3H), 3.07-3.13 (m, 2H), 1.75-1.80 (m, 2H), 0.99 (t, J=7.2 Hz, 3H); LC-MS: 449 (M+1).

### Example 21

### N-(2,4-difluoro-3-(8-methoxy-3H-pyrazolo[3,4-c]isoquinolin-7-yl)phenyl)-3-fluoropropane-1-sulfonamide

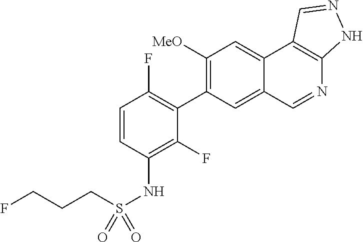 [SMILES: COc1cc2c(cnc3[nH]ncc32)cc1-c1c(F)ccc(NS(=O)(=O)CCCF)c1F]

The similar procedures of analogues C were followed to give the product. ¹HNMR (400 MHz, CDCl₃)□: δ 8.99 (s, 1H), 8.48 (s, 1H), 8.01 (s, 1H), 7.66-7.67 (m, 1H), 7.61 (s, 1H), 7.07-7.09 (m, 1H), 4.61-4.64 (m, 1H), 4.49-4.52 (m, 1H), 4.05 (s, 3H), 3.28-3.32 (m, 2H), 2.23-2.27 (m, 2H); LC-MS: 451 (M+H).

### Example 22

### N-(2-chloro-4-fluoro-3-(8-methoxy-3H-pyrazolo[3,4-c]isoquinolin-7-yl)phenyl)-3-fluoropropane-1-sulfonamide

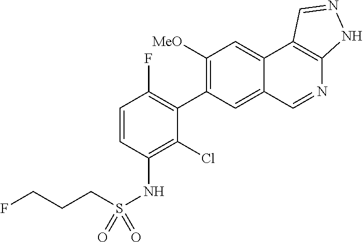 [SMILES: COc1cc2c(cnc3[nH]ncc32)cc1-c1c(F)ccc(NS(=O)(=O)CCCF)c1Cl]

The similar procedures of analogues C were followed to give the product. ¹HNMR (400 MHz, CDCl₃)□: δ 8.99 (s, 1H), 8.47 (s, 1H), 7.93 (s, 1H), 7.72-7.77 (m, 1H), 7.60 (s, 1H), 7.17-7.22 (m, 1H), 6.77 (brs, 1H), 4.62-4.65 (m, 1H), 4.51-4.53 (m, 1H), 4.01 (s, 3H), 3.28-3.32 (m, 2H), 2.23-2.28 (m, 2H); LC-MS: 467 (M+H).

### Example 23

### N-(3-(1-cyclopropyl-8-methoxy-3H-pyrazolo[3,4-c]isoquinolin-7-yl)-2,4-difluorophenyl)propane-1-sulfonamide

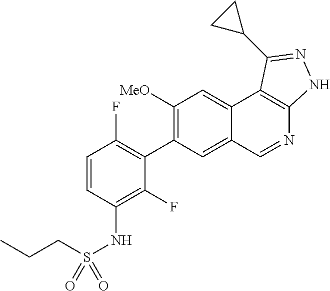 [SMILES: CCCS(=O)(=O)Nc1ccc(F)c(-c2cc3cnc4[nH]nc(C5CC5)c4c3cc2OC)c1F]

To a stirred solution of 2-(4-bromo-3-methoxyphenyl)acetonitrile (1 g, 4.43 mmol) in THF (10 mL) was added dropwise a solution of lithium bis(trimethylsilyl)amide (1 M solution in THF, 11 mL, 11.06 mmol) at −78° C. under nitrogen. The resulting solution was stirred at −78° C. for 15 min and then at room temperature for 1 hour. The reaction mixture was cooled to −78° C., and cyclopropanecarbonyl chloride (0.69 g, 6.637 mmol) was added dropwise. The mixture was allowed to warm to 0° C. over 1 hours and stirred at 0° C. for 1.5 hours. Saturated NH₄Cl was added and the organic layer was separated. The aqueous phase was extracted with ethyl acetate, and the combined organic phase were washed with brine, dried over Na₂SO₄ and concentrated. The residue was purified by column chromatography on silica gel to give 2-(4-bromo-3-methoxyphenyl)-3-cyclopropyl-3-oxopropanenitrile of 750 mg in 58% yield as yellow oil. LC-MS: 295 (M+1).

To a solution of 2-(4-bromo-3-methoxyphenyl)-3-cyclopropyl-3-oxopropanenitrile (750 mg, 2.54 mmol) in iso-propanol (15 mL), was added benzylhydrazine monohydrochloride (445 mg, 2.81 mmol), acetic acid (0.5 mL). The mixture was refluxed overnight. the solvents were removed in vacuo, the residue partitioned between ethyl acetate and saturated sodium bicarbonate, washed with brine, the organic layer was dried Na₂SO₄ and concentrated to give crude 1-benzyl-4-(4-bromo-3-methoxyphenyl)-3-cyclopropyl-1H-pyrazol-5-amine of 1 g as yellow solid, LCMS: 399 (M+1).

A mixture of 1-benzyl-4-(4-bromo-3-methoxyphenyl)-3-cyclopropyl-1H-pyrazol-5-amine (1 g, 2.51 mmol) and paraformaldehyde (82 mg, 2.78 mmol) in TFA (10 mL) was refluxed overnight. After removing most of TFA in vacuum, the residue was partitioned between ethyl acetate and saturated sodium bicarbonate, washed with brine. The organic layer was dried with Na₂SO₄, concentrated and purified by column chromatography on silica gel to give 3-benzyl-7-bromo-1-cyclopropyl-8-methoxy-3H-pyrazolo[3,4-c]isoquinoline of 0.9 g in 91% yield as yellow solid. ¹HNMR (400 MHz, DMSO-d6):□ δ 9.04 (s, 1H), 8.58 (s, 1H), 7.95 (s, 1H), 7.19-7.29 (m, 5H) 5.65 (s, 2H), 4.11 (s, 3H), 2.52-2.61 (m, 1H), 1.17-1.23 (m, 2H), 1.09-1.11 (m, 2H); LC-MS: 409 (M+1).

3-Benzyl-7-bromo-1-cyclopropyl-8-methoxy-3H-pyrazolo[3,4-c]isoquinoline (1 g, 2.45 mmol), bis(pinacolato)diboron (0.747 g, 2.94 mmol), potassium acetate (0.72 g, 7.35 mmol) and Pd(dppf)Cl₂ (179 mg) was dissolved in dioxane (30 ml). The mixture was evacuated and flushed with nitrogen for three times and then heated at 100° C. overnight. After cooled, filtered and washed with ethyl acetate, the combined organic layer was washed with brine, dried with Na₂SO₄ and concentrated. The residue was purified by flash chromatography to give 3-benzyl-1-cyclopropyl-8-methoxy-7-(4,4,5,5-tetramethyl-1,3,2-dioxaborolan-2-yl)-3H-pyrazolo[3,4-c]isoquinoline of 700 mg in 63% yield as yellow solid. LC-MS: 456 (M+1).

To a solution of 3-benzyl-1-cyclopropyl-8-methoxy-7-(4,4,5,5-tetramethyl-1,3,2-dioxaborolan-2-yl)-3H-pyrazolo[3,4-c]isoquinoline (50 mg, 0.109 mmol), N-(3-bromo-2,4-difluorophenyl)propane-1-sulfonamide (34 mg, 0.109 mol) in DME (3 mL) was added K₃PO₄.3H₂O (46 mg, 0.219 mmol) and dichlorobis[di-tert-butyl(4-dimethylaminophenyl)phosphino]palladium(II) (8 mg). The mixture was evacuated and flushed with nitrogen for three times and stirred at 110° C. for 5 hours under nitrogen. After cooled to room temperature, diluted with water and extracted with ethyl acetate. The organic layer was washed with brine, dried over Na₂SO₄ and concentrated. The residue was purified by PTLC to give N-(3-(3-benzyl-1-cyclopropyl-8-methoxy-3H-pyrazolo[3,4-c]isoquinolin-7-yl)-2,4-difluorophenyl)propane-1-sulfonamide of 30 mg 49% yield as yellow solid. LC-MS: 563 (M+1).

A mixture of N-(3-(3-benzyl-1-cyclopropyl-8-methoxy-3H-pyrazolo[3,4-c]isoquinolin-7-yl)-2,4-difluorophenyl)propane-1-sulfonamide (30 mg, 0.053 mmol), Pd(OH)₂ (60 mg, 20% on carbon), ammonium formate (8 mg) in formic acid (3 mL), was stirred at 100° C. for 7 hours. After cooled, filtered and washed with ethyl acetate, the solvents were removed in vacuum. The residue was partitioned between ethyl acetate and saturated sodium bicarbonate. The organic layer was washed with brine and dried with Na₂SO₄ and concentrated, the residue was purified by PTLC to give N-(3-(1-cyclopropyl-8-methoxy-3H-pyrazolo[3,4-c]isoquinolin-7-yl)-2,4-difluorophenyl)propane-1-sulfonamide of 6 mg in 24% yield as white solid. ¹HNMR (400 MHz, CDCl₃): δ 8.95 (s, 1H), 8.06 (s, 1H), 8.00 (s, 1H), 7.65-7.67 (m, 1H), 7.08-7.09 (m, 2H), 4.04 (s, 3H), 3.10-3.14 (m, 2H), 2.21-2.43 (m, 2H), 1.91-2.03 (m, 1H), 1.13-1.21 (m, 2H), 1.06 (t, J=7.5 Hz, 3H), 0.87-0.90 (m, 2H); LC-MS: 473 (M+1).

### Example 24

### N-(2-chloro-3-(1-cyclopropyl-8-methoxy-3H-pyrazolo[3,4-c]isoquinolin-7-yl)-4-fluorophenyl)propane-1-sulfonamide

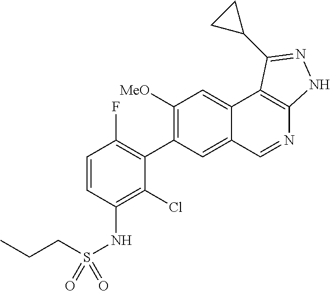 [SMILES: CCCS(=O)(=O)Nc1ccc(F)c(-c2cc3cnc4[nH]nc(C5CC5)c4c3cc2OC)c1Cl]

The similar procedures of analogues C were followed to give the product. ¹HNMR (400 MHz, CDCl₃): δ 8.95 (s, 1H), 8.06 (s, 1H), 7.93 (s, 1H) 7.65-7.67 (m, 1H), 7.16-7.21 (m, 2H), 6.76 (s, 1H), 4.01 (s, 3H) 3.11-3.15 (m, 2H), 2.21-2.43 (m, 2H), 1.91-2.03 (m, 1H), 1.13-1.21 (m, 2H), 1.06 (t, J=7.5 Hz, 3H), 0.87-0.90 (m, 2H); LC-MS: 490 (M+1).

### Example 25

### N-(2-chloro-3-(1-cyclopropyl-8-methoxy-3H-pyrazolo[3,4-c]isoquinolin-7-yl)-4-fluorophenyl)-3-fluoropropane-1-sulfonamide

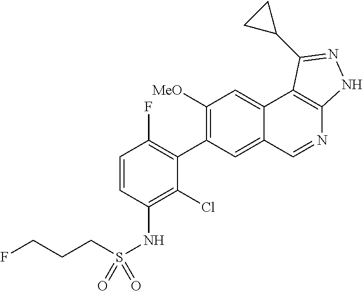 [SMILES: COc1cc2c(cnc3[nH]nc(C4CC4)c32)cc1-c1c(F)ccc(NS(=O)(=O)CCCF)c1Cl]

The similar procedures of analogues C were followed to give the product. ¹HNMR (400 MHz, CD₃OD)□: δ 8.97 (s, 1H), 8.14 (s, 1H), 8.05 (s, 1H), 7.65-7.69 (m, 1H), 7.23-7.27 (m, 1H), 4.61 (t, J=5.8 Hz, 1H), 4.49 (t, J=5.8 Hz, 1H), 4.09 (s, 3H), 3.26-3.28 (m, 2H), 2.51-2.54 (m, 1H), 2.01-2.24 (m, 2H), 1.18-1.20 (m, 2H), 1.06-1.07 (m, 2H); LC-MS: 507 (M+1).

### Example 26

### N-(3-(1-cyclopropyl-8-methoxy-3H-pyrazolo[3,4-c]isoquinolin-7-yl)-2,4-difluorophenyl)-3-fluoropropane-1-sulfonamide

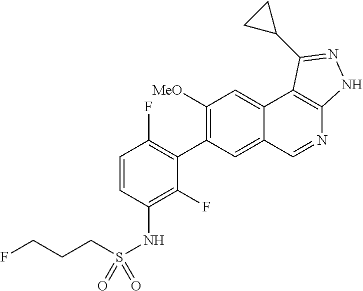 [SMILES: COc1cc2c(cnc3[nH]nc(C4CC4)c32)cc1-c1c(F)ccc(NS(=O)(=O)CCCF)c1F]

The similar procedures of analogues C were followed to give the product. LC-MS: 491 (M+1).

### Synthesis of Analogues D

### Example 27

### N-(2,4-difluoro-3-(3H-pyrrolo[2,3-c]isoquinolin-7-yl)phenyl)propane-1-sulfonamide

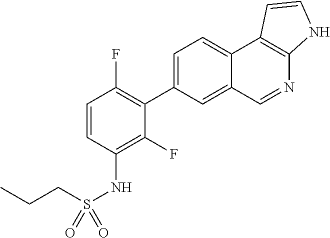 [SMILES: CCCS(=O)(=O)Nc1ccc(F)c(-c2ccc3c(cnc4[nH]ccc43)c2)c1F]

To 7-bromoisoquinolin-3-amine (256 mg) in DMF (4 mL) was added NIS (258 mg) in portions at ° C. The reaction mixture was stirred for 0.5 h. After adding water (20 mL) to quench the reaction, it was extracted with ethyl acetate, washed with water, brine and dried on Na₂SO₄, and concentrated. The residue was purified on a silica gel column to give 7-bromo-4-iodoisoquinolin-3-amine of 190 mg in 47% yield. LC-MS: 349 (M+1).

7-Bromo-4-iodoisoquinolin-3-amine (190 mg), Cul (5.2 mg), and (Ph₃P)₂PdCl₂ (19 mg) was dissolved in triethyl amine (20 mL). The mixture was evacuated and flushed with nitrogen for three times, followed by addition of the solution of trimethyl acetylene (80 mg) in triethyl amine (1 mL). The reaction mixture was stirred for 18 h at room temperature. After adding water (20 mL) to quench the reaction, it was extracted with ethyl acetate, washed with water, brine and dried on Na₂SO₄, and concentrated. The residue was purified on a silica gel column to give 7-bromo-4-((trimethylsilyl)ethynyl)isoquinolin-3-amine of 176 mg in 99% yield. LC-MS: 321 (M+1).

To 7-bromo-4-((trimethylsilyl)ethynyl)isoquinolin-3-amine (176 mg) in DCM (10 OmL) was added pyridine (87 mg) and acyl chloride (48 mg) at ° C. The reaction mixture was stirred for 4 hrs. It was extracted with ethyl acetate, washed with water, brine and dried on Na₂SO₄, and concentrated. The residue was purified on a silica gel column to give N-(7-bromo-4-((trimethylsilyl)ethynyl)isoquinolin-3-yl) acetamide of 110 mg in 55% yield. LC-MS: 361 (M+1).

N-(7-bromo-4-((trimethylsilyl)ethynyl)isoquinolin-3-yl) acetamide (110 mg) was dissolved in THF (5 mL), followed by the addition of TBAF (0.6 ml, 1N in THF). After the reaction mixture was refluxed for 1 h, it was cooled to room temperature and extracted with ethyl acetate, washed with water, brine and dried on Na₂SO₄, and concentrated to give the product of 7-bromo-3H-pyrrolo[2,3-c]isoquinoline (83 mg) in quantitative yield. ¹H NMR (400 MHz, CDCl₃) δ 9.23 (br, 1H), 8.83 (s, 1H), 8.20 (d, J=2.0 Hz, 1H), 8.09 (d, J=8.8 Hz, 1H), 7.81 (dd, J=8.8, 2.0 Hz, 1H), 7.38-7.40 (m, 1H), 6.99-7.00 (m, 1H); LC-MS: 247 (M+1).

7-Bbromo-3H-pyrrolo[2,3-c]isoquinoline (83 mg), bis(pinacolato)diboron (102 mg), potassium acetate (99 mg) and Pd(dppf)Cl₂ (25 mg) was dissolved in dioxane (4 ml). The mixture was evacuated and flushed with nitrogen for three times and then was heated at 80° C. overnight. It was cooled to room temperature and diluted with 100 mL ethyl acetate. After washed with water, brine and dried on Na₂SO₄, it was concentrated to give crude boronic ester. LC-MS: 295 (M+1).

Above crude boronic ester was mixed with N-(3-bromo-2,4-difluorophenyl)propane-1-sulfonamide (53 mg), tripotassium phosphate (71 mg) and Bis[di-tert-butyl-(4-dimethylaminophenyl)phosphine]dichloropalladium (2.4 mg) in the solution of dioxane (5 mL) and water (0.3 mL). The reaction mixture was evacuated and flushed with nitrogen for three times and then was heated at 110° C. for 5 h. It was cooled to room temperature and diluted with 100 mL ethyl acetate. After washed with water, brine and dried on Na₂SO₄ and concentrated. The residue was purified on a silica gel column to give N-(2,4-difluoro-3-(3H-pyrrolo[2,3-c]isoquinolin-7-yl)phenyl)propane-1-sulfonamide of 20 mg in 30% overall yields. ¹H NMR (400 MHz, CD₃SOCD₃) δ 12.10 (br, 1H), 9.71 (s, 1H), 8.97 (s, 1H), 8.39 (d, J=8.8 Hz, 1H), 8.24 (s, 1H), 7.79-7.81 (m, 1H), 7.40-7.52 (m, 2H), 7.26-7.29 (m, 1H), 7.08-7.10 (m, 1H), 3.11-3.15 (m, 2H), 1.76-1.79 (m, 2H), 0.99 (t, J=7.5 Hz, 3H). LC-MS: 402 (M+1).

### Synthesis of Analogues E

### Example 28

### N-(2,4-difluoro-3-(3H-imidazo[4,5-c]isoquinolin-7-yl)phenyl)propane-1-sulfonamide and its tautomer

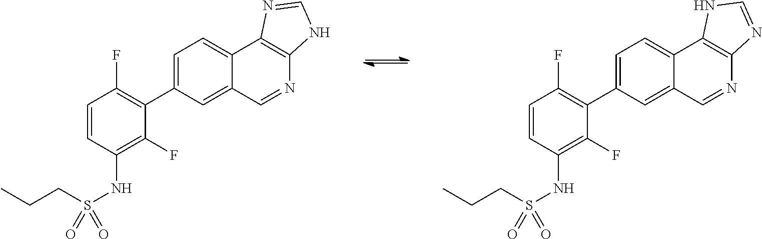 [SMILES: CCCS(=O)(=O)Nc1ccc(F)c(-c2ccc3c(cnc4[nH]cnc43)c2)c1F ; CCCS(=O)(=O)Nc1ccc(F)c(-c2ccc3c(cnc4nc[nH]c43)c2)c1F]

7-Bromoisoquinolin-3-amine (200 mg) was dissolved in conc. sulfuric acid (1 mL) at 0° C. Sodium nitrate (84 mg) was added in portions. The reaction mixture was stirred at room temperature for 0.5 h and then heated at 55° C. for 1 h. It was poured into iced water and basified to PH 9-10. After extracted with ethyl acetate (3×100 mL) and dried on Na₂SO₄, it was concentrated to give 7-bromo-4-nitroisoquinolin-3-amine without further purification.

To above 7-bromo-4-nitroisoquinolin-3-amine (241 mg) in DMF (6 mL) and conc. Hydrochloric acid (4 mL) was added tin chloride hydrate (712 mg). After the reaction mixture was stirred at 60° C. overnight, it was poured into iced water and basified to PH 9-10. After extracted with ethyl acetate (3×100 mL) and dried on Na₂SO₄, it was concentrated to give crude 7-bromoisoquinoline-3,4-diamine which was heated with excess triethyl orthofomate (6 mL) and acetic acid (0.5 mL) at 110° C. for 2 hs. After removing volatiles under decreased pressure, it gave 7-bromo-3H-imidazo[4,5-c]isoquinoline as yellow solid of 150 mg. LC-MS: 248 (M+1).

7-Bromo-3H-imidazo[4,5-c]isoquinoline (100 mg), bis(pinacolato)diboron (124 mg), potassium acetate (120 mg) and Pd(dppf)Cl₂ (15 mg) was mixed into a microwave tube. Dioxane (4 ml) was added. The mixture was evacuated and flushed with nitrogen for three times. The reaction was carried out in microwave wave conditions at 120° C. for 1 h. The reaction mixture was cooled to room temperature and diluted with 100 mL ethyl acetate. After washed with water, brine and dried on Na₂SO₄, it was concentrated to give crude boronic ester. LC-MS: 294 (M−1).

To above crude boronic ester intermediate in dry DCM (20 mL) was added acetic anhydride (62 mg) and triethyl amine (61 mg). The mixture was stirred at room temperature overnight. It was concentrated to give acyl protected boronic ester which mixed with N-(3-bromo-2,4-difluorophenyl)propane-1-sulfonamide (63 mg), potassium carbonate (55 mg) and Pd(dppf)Cl₂ (7 mg) in the solution of DMF (3 mL) and water (0.2 mL). The mixture was evacuated and flushed with nitrogen for three times. The reaction was carried out in microwave wave conditions at 140° C. for 1.5 h. The reaction mixture was cooled to room temperature and diluted with 100 mL ethyl acetate. After washed with water, brine and dried on Na₂SO₄, it was concentrated and purified on a silica gel column to give desired product of 13 mg in 13% overall yields. ¹H NMR (400 MHz, CD₃SOCD₃) δ 13.6 (br, 1H), 9.73 (s, 0.7H), 9.16 (s, 1H), 9.07 (s, 0.3H), 8.50 (s, 1H), 8.36 (s, 1H), 7.91-7.93 (m, 1H), 7.50-7.54 (m, 2H), 7.28-7.31 (m, 1H), 3.11-3.14 (m, 2H), 1.76-1.78 (m, 2H), 1.02 (t, J=7.5 Hz, 3H). LC-MS: 403 (M+1).

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the spirit and script of the invention, and all such variations are intended to be included within the scope of the following claims.

All references cited hereby are incorporated by reference in their entirety.

1. A compound of formula (I): 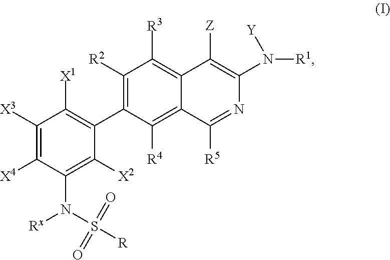
or a tautomer, a prodrug, or a pharmaceutically acceptable salt or solvate thereof, wherein:
Y is hydrogen or C₁-C₄ alkyl, and Z is selected from hydrogen, halogen, C₁-C₄ alkyl, C₁-C₄ haloalkyl, C₁-C₄ alkoxy, C₁-C₄ haloalkoxy, and —NR^{a}R^{b}; or alternatively, Y and Z are connected through a double bond (“Z═Y”) and are each independently CR^{y}, CR^{z}, or nitrogen (N), wherein R^{y} and R^{z} are each independently selected from hydrogen, halogen, hydroxyl, C₁-C₄ alkyl, C₃-C₆ cycloalkyl, C₁-C₄ haloalkyl, C₁-C₄ alkoxy, and C₁-C₄ haloalkoxy,
X¹, X², X³, and X⁴ are each independently selected from hydrogen, halogen, hydroxyl, C₁-C₄ alkyl, C₁-C₄ haloalkyl, C₁-C₄ alkoxy, and C₁-C₄ haloalkoxy;
R is selected from C₁-C₆ alkyl, C₃-C₆ cycloalkyl, C₆-C₁₀ aryl, and 5- to 10-membered heteroaryl, 5- to 10-membered heterocyclyl, C₃-C₆ cycloalkyl-(C₁-C₄)-alkyl, C₆-C₁₀ aryl-(C₁-C₄)-alkyl, 5- to 10-membered heteroaryl-(C₁-C₄)-alkyl, and 5- to 10-membered heterocyclyl-(C₁-C₄)-alkyl, each optionally substituted with one, two, or three substituents independently selected from halogen, hydroxyl, C₁-C₄ alkyl, C₁-C₄ haloalkyl, C₁-C₄ alkoxy, C₁-C₄ haloalkoxy, —NR^{c}R^{d}, cyano, nitro, oxo, —C(O)R⁶, —C(O)OR⁷, and —C(O)NR^{c}R^{d};
R^{x} is hydrogen or C₁-C₄ alkyl, or alternatively, R^{x} and R, together with the nitrogen (N) and sulfur (S) atoms to which they are attached, form a five- or six-membered ring;
R¹ is hydrogen, C₁-C₆ alkyl, C₆-C₁₀ aryl, benzyl, —C(O)R⁶, or —C(O)OR⁷, each optionally substituted with one, two or three substituents independent selected from halogen, C₁-C₄ alkyl, haloalkyl, C₁-C₄ alkoxy, C₁-C₄ alkoxy, cyano, and NR^{a}R^{b};
R², R³, R⁴, and R⁵ are each independently hydrogen, halogen, C₁-C₄ alkyl, C₁-C₄ haloalkyl, C₁-C₄ alkoxy, and C₁-C₄ haloalkoxy;
R^{a} and R^{b} are each independently selected from hydrogen, C₁-C₆ alkyl, benzyl, and —C(O)OR⁷, and
R⁶ is hydrogen or C₁-C₄ alkyl;
R⁷ is C₁-C₄ alkyl; and
R^{c} and R^{d} are each independently hydrogen or C₁-C₄ alkyl.

2. The compound of claim 1, or a tautomer, a prodrug, or a pharmaceutically acceptable salt or solvate thereof, wherein Y is hydrogen or C₁-C₄ alkyl, and Z is selected from hydrogen, halogen, C₁-C₄ alkyl, C₁-C₄ haloalkyl, C₁-C₄ alkoxy, C₁-C₄ haloalkoxy, and —NR^{a}R^{b}.

3. The compound of claim 2, or a tautomer, a prodrug, or a pharmaceutically acceptable salt or solvate thereof, wherein Y is hydrogen, and Z is hydrogen.

4. The compound of claim 1, or a tautomer, a prodrug, or a pharmaceutically acceptable salt or solvate thereof, wherein R¹ is hydrogen or C₁-C₆ alkyl optionally substituted with —NR^{a}R^{b}, wherein R^{a} and R^{b} are independently selected from hydrogen and —C(O)OR⁷.

5. The compound of claim 4, or a tautomer, a prodrug, or a pharmaceutically acceptable salt or solvate thereof, wherein R is selected from C₁-C₆ alkyl, C₃-C₆ cycloalkyl, and C₆-C₁₀ aryl, each optionally substituted with one, two, or three substituents independently selected from halogen, C₁-C₄ alkyl, C₁-C₄ haloalkyl, C₁-C₄ alkoxy, and C₁-C₄ haloalkoxy.

6. The compound of claim 5, or a tautomer, a prodrug, or a pharmaceutically acceptable salt or solvate thereof, wherein X¹, X², X³, and X⁴ are independently hydrogen or halogen; R², R³, R⁴, and R⁵ are each independently hydrogen, C₁-C₄ alkyl, C₁-C₄ alkoxy, or C₁-C₄ haloalkoxy; and R is C₁-C₆ alkyl optionally substituted with one, two, or three substituents independently selected from halogen and C₁-C₄ alkoxy.

7. The compound of claim 2, or a tautomer, a prodrug, or a pharmaceutically acceptable salt or solvate thereof, wherein:
Y and Z are each hydrogen;
X¹ and X² are each independently fluoro (F) or chloro (Cl);
X³ and X⁴ are each hydrogen;
R¹ is hydrogen or C₁-C₆ alkyl optionally substituted by —NHCOOR⁷, wherein R⁷ is C₁-C₄ alkyl;
R² is hydrogen, C₁-C₄ alkoxy, or C₁-C₄ haloalkoxy;
R³, R⁴, and R⁵ are each hydrogen;
R^{x} is hydrogen; and
R is C₁-C₆ alkyl optionally substituted by one to three halogen atoms.

8. The compound of claim 2, or a tautomer, a prodrug, or a pharmaceutically acceptable salt or solvate thereof, selected from the group consisting of:
N-[3-(3-amino-7-isoquinolyl)-2,4-difluoro-phenyl]propane-1-sulfonamide;
methyl N-[(1S)-2-[[7-[2,6-difluoro-3-(propylsulfonylamino)phenyl]-3-isoquinolyl]amino]-1-methyl-ethyl]carbamate;
N-[3-(3-amino-6-methoxy-7-isoquinolyl)-2,4-difluoro-phenyl]propane-1-sulfonamide;
methyl N-[(1S)-2-[[7-[2,6-difluoro-3-(propylsulfonylamino)phenyl]-6-methoxy-3-isoquinolyl]amino]-1-methyl-ethyl]carbamate;
methyl N-[(1R)-2-[[7-[2,6-difluoro-3-(propylsulfonylamino)phenyl]-6-methoxy-3-isoquinolyl]amino]-1-methyl-ethyl]carbamate;
methyl N-[(1S)-2-[[7-[2,6-difluoro-3-(propylsulfonylamino)phenyl]-6-(2-fluoroethoxy)-3-isoquinolyl]amino]-1-methyl-ethyl]carbamate; and
methyl N-[(1S)-2-[[7-[2,6-difluoro-3-(propylsulfonylamino)phenyl]-6-ethyl-3-isoquinolyl]amino]-1-methyl-ethyl]carbamate.

9. The compound of claim 1, wherein Z and Y are connected through a double bond (Z═Y) and are each independently CR^{y}, CR^{z}, or nitrogen (N), further characterized by formula (II): 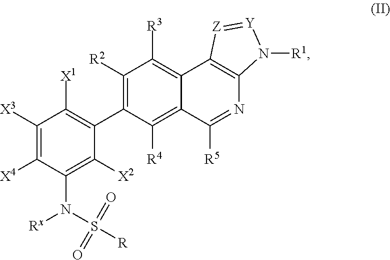
or a tautomer, a prodrug, or a pharmaceutically acceptable salt or solvate thereof.

10. The compound of claim 9, or a tautomer, a prodrug, or a pharmaceutically acceptable salt or solvate thereof, wherein R¹ is hydrogen, —C(O)R⁶, or C₁-C₆ alkyl optionally substituted with —NR^{a}R^{b}, wherein R^{a} and R^{b} are independently selected from hydrogen and —C(O)OR.

11. The compound of claim 10, or a tautomer, a prodrug, or a pharmaceutically acceptable salt or solvate thereof, wherein R is selected from C₁-C₆ alkyl, C₃-C₆ cycloalkyl, and C₆-C₁₀ aryl, each optionally substituted with one, two, or three substituents independently selected from halogen, C₁-C₄ alkyl, C₁-C₄ haloalkyl, C₁-C₄ alkoxy, and C₁-C₄ haloalkoxy.

12. The compound of claim 9, or a tautomer, a prodrug, or a pharmaceutically acceptable salt or solvate thereof, wherein X¹, X², X³, and X⁴ are independently hydrogen or halogen; R², R³, R⁴, and R⁵ are each independently hydrogen, C₁-C₄ alkyl, C₁-C₄ alkoxy, C₁-C₄ haloalkoxy, and R is C₁-C₆ alkyl optionally substituted with one, two, or three substituents independently selected from halogen and C₁-C₄ alkoxy.

13. The compound of claim 10, or a tautomer, a prodrug, or a pharmaceutically acceptable salt or solvate thereof, wherein R^{x} and R, together with the nitrogen (N) and sulfur (S) atoms to which they are attached, form a five- or six-membered ring.

14. The compound of claim 10, or a tautomer, a prodrug, or a pharmaceutically acceptable salt or solvate thereof, wherein R^{x} and R together form —CH₂CH₂CH₂—.

15. The compound of claim 9, or a tautomer, a prodrug, or a pharmaceutically acceptable salt or solvate thereof, wherein:
X¹ and X² are each independently fluoro (F) or chloro (Cl);
X³ and X⁴ are each hydrogen;
R¹ is hydrogen or C₁-C₆ alkyl optionally substituted by —NHCOOR⁷, wherein R⁷ is C₁-C₄ alkyl;
R² is hydrogen, C₁-C₄ alkoxy, or C₁-C₄ haloalkoxy;
R³, R⁴, and R⁵ are each hydrogen;
R^{x} is hydrogen;
R is C₁-C₆ alkyl optionally substituted by one to three halogen atoms;
R^{y} and R^{z} are each independently selected from hydrogen, halogen, C₁-C₄ alkyl, and C₃-C₆ cycloalkyl.

16. The compound of claim 9, wherein Y is nitrogen (N) and Z is C—R^{z}, further characterized by formula (IIa): 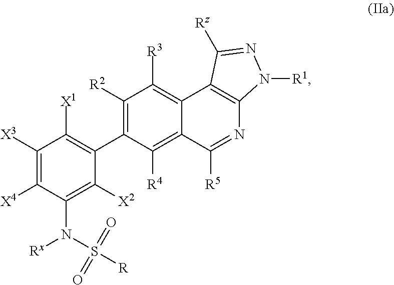
or a tautomer, a prodrug, or a pharmaceutically acceptable salt or solvate thereof, wherein R^{z} is selected from hydrogen, halogen, C₁-C₄ alkyl, C₃-C₆ cycloalkyl, C₁-C₄ haloalkyl, C₁-C₄ alkoxy, and NR^{a}R^{b}.

17. The compound of claim 16, or a tautomer, a prodrug, or a pharmaceutically acceptable salt or solvate thereof, wherein R¹ is hydrogen, —C(O)R⁶, or C₁-C₆ alkyl optionally substituted with —NR^{a}R^{b}, wherein R^{a} and R^{b} are independently selected from hydrogen and —C(O)OR⁷.

18. The compound of claim 17, or a tautomer, a prodrug, or a pharmaceutically acceptable salt or solvate thereof, wherein R is selected from C₁-C₆ alkyl, C₃-C₆ cycloalkyl, and C₆-C₁₀ aryl, each optionally substituted with one, two, or three substituents independently selected from halogen, C₁-C₄ alkyl, C₁-C₄ haloalkyl, C₁-C₄ alkoxy, and C₁-C₄ haloalkoxy.

19. The compound of claim 16, or a tautomer, a prodrug, or a pharmaceutically acceptable salt or solvate thereof, wherein X¹, X², X³, and X⁴ are independently hydrogen or halogen; R², R³, R⁴, and R⁵ are each independently hydrogen, C₁-C₄ alkyl, C₁-C₄ alkoxy, or C₁-C₄ haloalkoxy, and R is C₁-C₆ alkyl optionally substituted with one, two, or three substituents independently selected from halogen and C₁-C₄ alkoxy.

20. The compound of claim 17, or a tautomer, a prodrug, or a pharmaceutically acceptable salt or solvate thereof, wherein R^{x} and R, together with the nitrogen (N) and sulfur (S) atoms to which they are attached, form a five- or six-membered ring.

21. The compound of claim 17, or a tautomer, a prodrug, or a pharmaceutically acceptable salt or solvate thereof, wherein R^{x} and R together form —CH₂CH₂CH₂—.

22. The compound of claim 16, or a tautomer, a prodrug, or a pharmaceutically acceptable salt or solvate thereof, wherein:
X¹ and X² are each independently fluoro (F) or chloro (Cl);
X³ and X⁴ are each hydrogen;
R¹ is hydrogen or C₁-C₆ alkyl optionally substituted by —NHCOOR⁷, wherein R⁷ is C₁-C₄ alkyl;
R² is hydrogen, C₁-C₄ alkoxy, or C₁-C₄ haloalkoxy;
R³, R⁴, and R⁵ are each hydrogen;
R^{x} is hydrogen;
R is C₁-C₆ alkyl optionally substituted by one to three halogen atoms;
R^{z} is selected from hydrogen, halogen, C₁-C₄ alkyl, and C₃-C₆ cycloalkyl.

23. The compound of claim 9, wherein Y is C—R^{y} and Z is nitrogen (N), further characterized by formula (IIb): 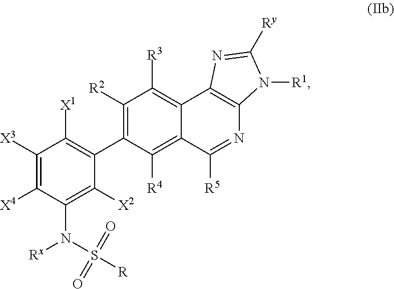
or a tautomer, a prodrug, or a pharmaceutically acceptable salt or solvate thereof, wherein R^{y} is selected from hydrogen, halogen, C₁-C₄ alkyl, C₃-C₆ cycloalkyl, C₁-C₄ haloalkyl, C₁-C₄ alkoxy, and NR^{a}R^{b}.

24. The compound of claim 23, or a tautomer, a prodrug, or a pharmaceutically acceptable salt or solvate thereof, wherein R¹ is hydrogen, —C(O)R⁶, or C₁-C₆ alkyl optionally substituted with —NR^{a}R^{b}, wherein R^{a} and R^{b} are independently selected from hydrogen and —C(O)OR⁷.

25. The compound of claim 24, or a tautomer, a prodrug, or a pharmaceutically acceptable salt or solvate thereof, wherein R is selected from C₁-C₆ alkyl, C₃-C₆ cycloalkyl, and C₆-C₁₀ aryl, each optionally substituted with one, two, or three substituents independently selected from halogen, C₁-C₄ alkyl, C₁-C₄ haloalkyl, C₁-C₄ alkoxy, and C₁-C₄ haloalkoxy.

26. The compound of claim 23, or a tautomer, a prodrug, or a pharmaceutically acceptable salt or solvate thereof, wherein X¹, X², X³, and X⁴ are independently hydrogen or halogen; R², R³, R⁴, and R⁵ are each independently hydrogen, C₁-C₄ alkyl, C₁-C₄ alkoxy, C₁-C₄ haloalkoxy, and R is C₁-C₆ alkyl optionally substituted with one, two, or three substituents independently selected from halogen and C₁-C₄ alkoxy.

27. The compound of claim 23, or a tautomer, a prodrug, or a pharmaceutically acceptable salt or solvate thereof, wherein:
X¹ and X² are each independently fluoro (F) or chloro (Cl);
X³ and X⁴ are each hydrogen;
R¹ is hydrogen or C₁-C₆ alkyl optionally substituted by —NHCOOR⁷, wherein R⁷ is C₁-C₄ alkyl;
R² is hydrogen, C₁-C₄ alkoxy, or C₁-C₄ haloalkoxy;
R³, R⁴, and R⁵ are each hydrogen;
R^{x} is hydrogen;
R is C₁-C₆ alkyl optionally substituted by one to three halogen atoms;
R^{y} is selected from hydrogen, halogen, C₁-C₄ alkyl, and C₃-C₆ cycloalkyl.

28. The compound of claim 9, wherein Y is C—R^{y} and Z is C—R^{z}, further characterized by formula (IIc): 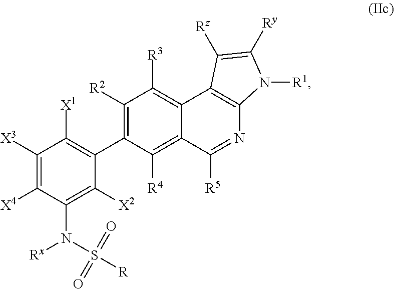
or a tautomer, a prodrug, or a pharmaceutically acceptable salt or solvate thereof, wherein R^{y} and R^{z} are each independently selected from hydrogen, halogen, C₁-C₄ alkyl, C₃-C₆ cycloalkyl, C₁-C₄ haloalkyl, C₁-C₄ alkoxy, and NR^{a}R^{b}.

29. The compound of claim 28, or a tautomer, a prodrug, or a pharmaceutically acceptable salt or solvate thereof, wherein R¹ is hydrogen, —C(O)R⁶, or C₁-C₆ alkyl optionally substituted with —NR^{a}R^{b}, wherein R^{a} and R^{b} are independently selected from hydrogen and —C(O)OR⁷.

30. The compound of claim 29, or a tautomer, a prodrug, or a pharmaceutically acceptable salt or solvate thereof, wherein R is selected from C₁-C₆ alkyl, C₃-C₆ cycloalkyl, and C₆-C₁₀ aryl, each optionally substituted with one, two, or three substituents independently selected from halogen, C₁-C₄ alkyl, C₁-C₄ haloalkyl, C₁-C₄ alkoxy, and C₁-C₄ haloalkoxy.

31. The compound of claim 28, or a tautomer, a prodrug, or a pharmaceutically acceptable salt or solvate thereof, wherein X¹, X², X³, and X⁴ are independently hydrogen or halogen; R², R³, R⁴, and R⁵ are each independently hydrogen, C₁-C₄ alkyl, C₁-C₄ alkoxy, C₁-C₄ haloalkoxy, and R is C₁-C₆ alkyl optionally substituted with one, two, or three substituents independently selected from halogen and C₁-C₄ alkoxy.

32. The compound of claim 28, or a tautomer, a prodrug, or a pharmaceutically acceptable salt or solvate thereof, wherein:
X¹ and X² are each independently fluoro (F) or chloro (Cl);
X³ and X⁴ are each hydrogen;
R¹ is hydrogen or C₁-C₆ alkyl optionally substituted by —NHCOOR⁷, wherein R⁷ is C₁-C₄ alkyl;
R² is hydrogen, C₁-C₄ alkoxy, or C₁-C₄ haloalkoxy;
R³, R⁴, and R⁵ are each hydrogen;
R^{x} is hydrogen;
R is C₁-C₆ alkyl optionally substituted by one to three halogen atoms;
R^{y} and R^{z} are each independently selected from hydrogen, halogen, C₁-C₄ alkyl, and C₃-C₆ cycloalkyl.

33. The compound of claim 1, or a tautomer, a prodrug, a pharmaceutically acceptable salt or solvate thereof, selected from the group consisting of:
N-[2,4-difluoro-3-(3H-pyrazolo[3,4-c]isoquinolin-7-yl)phenyl]propane-1-sulfonamide;
N-[3-(1-bromo-3H-pyrazolo[3,4-c]isoquinolin-7-yl)-2,4-difluoro-phenyl]propane-1-sulfonamide;
N-[3-(1-cyclopropyl-3H-pyrazolo[3,4-c]isoquinolin-7-yl)-2,4-difluoro-phenyl]propane-1-sulfonamide;
N-[2-chloro-4-fluoro-3-(3H-pyrazolo[3,4-c]isoquinolin-7-yl)phenyl]propane-1-sulfonamide;
N-[3-(1-bromo-3H-pyrazolo[3,4-c]isoquinolin-7-yl)-2-chloro-4-fluoro-phenyl]propane-1-sulfonamide;
N-[2-chloro-3-(1-cyclopropyl-3H-pyrazolo[3,4-c]isoquinolin-7-yl)-4-fluoro-phenyl]propane-1-sulfonamide;
N-[2,4-difluoro-3-(3H-pyrazolo[3,4-c]isoquinolin-7-yl)phenyl]-3-fluoro-propane-1-sulfonamide;
N-[2-chloro-4-fluoro-3-(3H-pyrazolo[3,4-c]isoquinolin-7-yl)phenyl]-3-fluoro-propane-1-sulfonamide;
N-[2,4-dichloro-3-(3H-pyrazolo[3,4-c]isoquinolin-7-yl)phenyl]propane-1-sulfonamide;
N-[4-chloro-2-fluoro-3-(3H-pyrazolo[3,4-c]isoquinolin-7-yl)phenyl]propane-1-sulfonamide;
2-[2,4-difluoro-3-(3H-pyrazolo[3,4-c]isoquinolin-7-yl)phenyl]-1,2-thiazolidine 1,1-dioxide;
N-[2,4-difluoro-3-(8-methoxy-3H-pyrazolo[3,4-c]isoquinolin-7-yl)phenyl]propane-1-sulfonamide;
N-[2-chloro-4-fluoro-3-(8-methoxy-3H-pyrazolo[3,4-c]isoquinolin-7-yl)phenyl]propane-1-sulfonamide;
N-[2,4-difluoro-3-(8-methoxy-3H-pyrazolo[3,4-c]isoquinolin-7-yl)phenyl]-3-fluoro-propane-1-sulfonamide;
N-[2-chloro-4-fluoro-3-(8-methoxy-3H-pyrazolo[3,4-c]isoquinolin-7-yl)phenyl]-3-fluoro-propane-1-sulfonamide;
N-[3-(1-cyclopropyl-8-methoxy-3H-pyrazolo[3,4-c]isoquinolin-7-yl)-2,4-difluoro-phenyl]propane-1-sulfonamide;
N-[2-chloro-3-(1-cyclopropyl-8-methoxy-3H-pyrazolo[3,4-c]isoquinolin-7-yl)-4-fluoro-phenyl]propane-1-sulfonamide;
N-[2-chloro-3-(1-cyclopropyl-8-methoxy-3H-pyrazolo[3,4-c]isoquinolin-7-yl)-4-fluoro-phenyl]-3-fluoro-propane-1-sulfonamide;
N-[3-(1-cyclopropyl-8-methoxy-3H-pyrazolo[3,4-c]isoquinolin-7-yl)-2,4-difluoro-phenyl]-3-fluoro-propane-1-sulfonamide;
N-[2,4-difluoro-3-(3H-pyrrolo[2,3-c]isoquinolin-7-yl)phenyl]propane-1-sulfonamide; and
N-[2,4-difluoro-3-(3H-imidazo[4,5-c]isoquinolin-7-yl)phenyl]propane-1-sulfonamide.

34. A composition comprising a compound of claim 1, or a tautomer, a prodrug, or a pharmaceutically acceptable salt or solvate thereof, and a pharmaceutically acceptable carrier.

35. A method of treating a hyperproliferative disease or disorder, comprising administering to a mammalian patient in need thereof a therapeutically effective amount of a compound of claim 1, or a tautomer, a prodrug, or a pharmaceutically acceptable salt or solvate thereof.

36. A method of treating a hyperproliferative disease or disorder, comprising administering to a mammalian patient in need thereof a composition of claim 34.

37. The method of claim 35, wherein the hyperproliferative disease or disorder is associated with BRAF^{V600E} kinase activity.

38. The method of claim 35, wherein the hyperproliferative disease or disorder is a cancer.

39. The method of claim 35, wherein the hyperproliferative disease or disorder is selected from melanomas; papillary thyroid, colorectal, ovarian, breast, and lung cancers; and leukemia.

40. The method of claim 35, further in conjunction with administering to the patient a therapeutically effective amount of a second therapeutic agent.

41. The method of claim 40, wherein the second therapeutic agent is a different anticancer agent.

42-45. (canceled)

46. An in vitro method of modulating BRAF^{V600E} kinase activity, the method comprising contacting a tissue culture comprising BRAF^{V600E} kinase with a compound of claim 1.

47. (canceled)
